(19) United States
(12) Patent Application Publication
(10) Pub. No.: US 2002/0025968 A1
(43) Pub. Date: Feb. 28, 2002

(54) Method for inhibiting neoplastic cells and related conditions by exposure to 4-aminoquinazoline derivatives

(76) Inventors: Rifat Pamukcu, Spring House, PA (US); Gary Piazza, Doylestown, PA (US)

Correspondence Address: Robert W. Stevenson, Cell Pathways, Inc., 702 Electronic Drive, Horsham, PA, 19044, US

(21) Appl. No.: 09/952,769

(22) Filed: Sep. 14, 2001

Related U.S. Application Data
(63) Continuation of application No. 09/060,444, filed on Apr. 15, 1998, now abandoned.

(51) Int. Cl. A61K 31/517

(52) U.S. Cl. 514/259

(57) ABSTRACT

A method for inhibiting neoplastic cells and related conditions by exposing them to 4-aminoquinazoline derivatives.

This application is a Continuation of prior U.S. application Ser. No. 09/060,444 filed Apr. 15, 1998 entitled “Method for Inhibiting Neoplastic Cells and Related Conditions by Exposure to 4-Aminoquinazoline Derivatives” which is incorporated herein by reference.

### TECHNICAL FIELD

This invention relates to a method for the selective inhibition of neoplastic cells, for example, for the treatment or prevention of precancerous lesions or other neoplasias in mammals.

### BACKGROUND OF THE INVENTION

Each year in the United States alone, untold numbers of people develop precancerous lesions, which is a form of neoplasia, as discussed below. Such lesions exhibit a strong tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms. Compounds that prevent or induce the remission of existing precancerous or cancerous lesions or carcinomas would greatly reduce illness and death from cancer.

For example, approximately 60,000 people die from colon cancer, and over 150,000 new cases of colon cancer are diagnosed each year. For the American population as a whole, individuals have a six percent lifetime risk of developing colon cancer, making it the second most prevalent form of cancer in the country. Colon cancer is also prevalent in Western Europe. It is believed that increased dietary fat consumption is increasing the risk of colon cancer in Japan.

In addition, the incidence of colon cancer reportedly increases with age, particularly after the age of 40. Since the mean ages of populations in America and Western Europe are increasing, the prevalence of colorectal cancer should increase in the future.

To date, little progress has been made in the prevention and treatment of colorectal cancer, as reflected by the lack of change in the five-year survival rate over the last few decades. The only cure for this cancer is surgery at an extremely early stage. Unfortunately, most of these cancers are discovered too late for surgical cure. In many cases, the patient does not experience symptoms until the cancer has progressed to a malignant stage.

In view of these grim statistics, efforts in recent years have concentrated on colon cancer prevention. Colon cancer usually arises from pre-existing benign neoplastic growths known as polyps. Prevention efforts have emphasized the identification and removal of colonic polyps. Polyps are identified by x-ray and/or colonoscopy, and usually removed by devices associated with the colonoscope. The increased use of colon x-rays and colonoscopies in recent years has detected clinically significant precancerous polyps in four to six times the number of individuals per year that acquire colon cancer. During the past five years alone, an estimated 3.5 to 5.5 million people in the United States have been diagnosed with adenomatous colonic polyps, and it is estimated that many more people have or are susceptible to developing this condition, but are as yet undiagnosed. In fact, there are estimates that 10-12 percent of people over the age of 40 will form clinically significant adenomatous polyps.

Removal of polyps has been accomplished either with surgery or fiber-optic endoscopic polypectomy—procedures that are uncomfortable, costly (the cost of a single polypectomy ranges between $1,000 and $1,500 for endoscopic treatment and more for surgery), and involve a small but significant risk of colon perforation. Overall, about $2.5 billion is spent annually in the United States in colon cancer treatment and prevention.

In the breast, breast cancer is often treated surgically, often by radical mastectomy with its painful aftermath. Such surgery is costly, too.

As indicated above, each lesion carries with it a chance that it will develop into a cancer. The likelihood of cancer is diminished if a precancerous lesion is removed. However, many of these patients demonstrate a propensity for developing additional lesions in the future. They must, therefore, be monitored periodically for the rest of their lives for reoccurrence.

In most cases (i.e. the cases of sporadic lesion formation, e.g. so-called common sporadic polyps), lesion removal will be effective to reduce the risk of cancer. In a small percentage of cases (i.e. cases where numerous lesions form, e.g. the so-called polyposis syndromes), removal of all or part of the effected area (e.g. the colon) is indicated. For example, the difference between common sporadic polyps and polyposis syndromes is dramatic. Common sporadic polyp cases are characterized by relatively few polyps which can usually be removed leaving the colon intact. By contrast, polyposis syndrome cases can be characterized by many (e.g. hundreds or more) of polyps—literally covering the colon in some cases—making safe removal of the polyps impossible short of surgical removal of the colon.

Because each lesion carries with it a palpable risk of cancerous development, patients who form many lesions (e.g. polyposis syndrome patients) invariably develop cancer if left untreated. Surgical removal of the colon is the conventional treatment in polyposis patients. Many polyposis patients have undergone a severe change in lifestyle as a result of the disfiguring surgery. Patients have strict dietary restrictions, and many must wear ostomy appliances to collect their intestinal wastes.

The search for drugs useful for treating and preventing cancer is intensive. Indeed, much of the focus of cancer research today is on the prevention of cancer because chemotherapy for cancer itself is often not effective and has severe side effects. Cancer chemoprevention is important for recovered cancer patients who retain a risk of cancer reoccurrence. Also, cancer prevention is important for people who have not yet had cancer, but have hereditary factors that place them at risk of developing cancer. With the development of new genetic screening technologies, it is easier to identify those patients with high-risk genetic factors, such as the potential for polyposis syndrome, who would greatly benefit from chemopreventative drugs. Therefore, finding such anti-cancer drugs that can be used for prolonged preventive use is of vital interest.

Known chemopreventative and chemotherapeutic drugs are believed to kill cancer cells by inducing apoptosis, or as sometimes referred to as “programmed cell death.” Apoptosis naturally occurs in virtually all tissues of the body, and especially in self-renewing tissues such as bone marrow, gut, and skin. Apoptosis plays a critical role in tissue homeostasis, that is, it ensures that the number of new cells produced are correspondingly offset by an equal number of cells that die. For example, the cells in the intestinal lining divide so rapidly that the body must eliminate cells after only three days in order to prevent the overgrowth of the intestinal lining.

Recently, scientists have realized that abnormalities of apoptosis can lead to the formation of precancerous lesions and carcinomas. Also, recent research indicates that defects in apoptosis play a major role in other diseases in addition to cancer. Consequently, compounds that modulate apoptosis could be used to prevent or control cancer, as well as used in the treatment of other diseases.

Unfortunately, even though known chemotherapeutic drugs may exhibit such desirable apoptosis effects, most chemotherapeutic drugs have serious side effects that prohibit their long-term use, or use in otherwise healthy individuals with precancerous lesions. These side effects, which are a result of the high levels of cytotoxicity of the drugs, include hair loss, weight loss, vomiting, immune suppression and other toxicities. Therefore, there is a need to identify new drug candidates for therapy that do not have such serious side effects in humans.

In recent years, several non-steroidal anti-inflammatory drugs (“NSAIDs”), originally developed to treat arthritis, have shown effectiveness in inhibiting and eliminating colonic polyps. Polyps virtually disappear when the patients take the drug, particularly when the NSAID sulindac is administered. However, the prophylactic use of currently available NSAIDs, even in polyposis syndrome patients, is marked by severe side reactions that include gastrointestinal irritations and ulcerations. Once NSAID treatment is terminated due to such complications, the polyps return, particularly in polyposis syndrome patients.

Sulindac has been particularly well received among the NSAIDs for the polyp treatment. Sulindac is a sulfoxide compound that itself is believed to be inactive as an anti-arthritic agent. The sulfoxide is reportedly converted by liver enzymes to the corresponding sulfide, which is acknowledged to be the active moiety as a prostaglandin synthesis inhibitor. The sulfide, however, is associated with the side effects of conventional NSAIDs. The sulfoxide is also known to be metabolized to sulfone compound that has been found to be inactive as an inhibitor of prostaglandin synthesis but active as an inhibitor of precancerous lesions.

### SUMMARY OF THE INVENTION

This invention includes a method of inhibiting neoplastic cells by exposing those cells to a pharmacologically effective amount of those compounds described below. Such compounds are effective in modulating apoptosis and eliminating and inhibiting the growth of neoplasias such as precancerous lesions.

The compounds that are useful in the methods of this invention include those of Formula I:

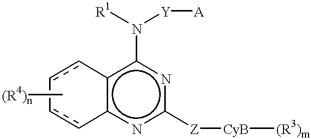

wherein

R₁ is hydrogen or C1-4 alkyl;

Y is C1-6 alkylene; A is -O-R₀ or -S(O)ₚ-R₀,

R₀ is C1-4 alkyl-hydroxy;

p is 0-2;

Z is single bond, methylene, ethylene (CH₂CH₂), vinylene (CH═CH) or ethynylene (C═C);

CyB is:

(1) 7-membered, unsaturated or partially saturated, monocyclic hetero ring containing as hetero atoms, one, two or three nitrogen atoms,

(2) 6-membered, unsaturated or partially saturated, monocyclic hetero ring containing as hetero atoms, two or three nitrogen atoms,

(3) 6-membered, unsaturated or partially saturated, monocyclic hetero ring containing as hetero atoms, one nitrogen atom,

(4) 4-or 5-membered, unsaturated or partially saturated, monocyclic hetero ring containing as hetero atoms, one, two or three nitrogen atoms, or

(5) 4-7 membered, unsaturated or partially saturated, monocyclic hetero ring containing as hetero atoms, one or two oxygen atoms, or one or two sulfur atoms;

R₃ is hydrogen, C1-4 alkyl, C1-4 alkoxy, halogen or trifluoromethyl;

R₄ is (1) hydrogen, (2) C1-4 alkyl, (3) C1-4 alkoxy, (4) —COOR₈, in which R₈ is hydrogen or C1-4 alkyl, (5) —NR₉R₁₀, (6) —NHCOR₁₁, (7) —NHSO₂R₁₁, (8) SO₂NR₉R₁₀, (9) —OCOR₁₁, (10) halogen, (11) trifluoromethyl, (12) hydroxy, (13) nitro, (14) cyano, (15) —SO₂N═CHN R₁₁R₁₀, (16) —CONR₁₂R₁₃, (17) C1-4 alkylthio, (18) C1-4 alkylsulfinyl, (19) C1 -4 alkylsulfonyl, (20) ethynyl, (21) hydroxymethyl, (22) tri(C1-4 alkyl) silylethynyl or (23) acetyl; and m and n independently are 1 or 2; with the proviso that a CyB ring should not bond to Z through a nitrogen atom in the CyB ring when Z is vinylene or ethynylene;

R₉ is hydrogen, C1-4 alkyl or phenyl(C1-4 alkyl);

R₁₀ is hydrogen or C1-4 alkyl;

R₁₁ is C1-4 alkyl;

R₁₂ is hydrogen or C1-4 alkyl;

R₁₃ is C1-4 alkyl or phenyl(C1-4 alkyl);

or pharmaceutically acceptable acid addition salts, pharmaceutically acceptable salts, or hydrates thereof.

### DETAILED DESCRIPTION OF THE INVENTION

As discussed above, this invention involves the use of compounds described above for treating patients with neoplasia, for example cancerous or precancerous lesions.

As used herein, the term “C 1-4 alkyl” means methyl, ethyl, propyl, butyl and the isomers thereof. The term “C1-4 alkyl-hydroxy” means 1 -hydroxymethyl, 2-hydroxyethyl, 3-hydroxypropyl, 4-hydroxybutyl and the isomers thereof. The term “C1-4 alkoxy” means methoxy, ethoxy, propoxy, butoxy and isomers thereof.

As used herein the term “halogen” means fluorine, chlorine, bromine and iodine. The term “C1-6 alkylene” means methylene, ethylene, trimethylene, tetramethylene, pentamethylene, hexamethylene and isomers thereof.

In Formula (I), examples of 7-membered, unsaturated or partially saturated, monocyclic hetero ring containing as hetero atoms, one, two or three nitrogen atoms, represented by CyB-(1), are azepine, diazepine, triazepine, and partially saturated rings thereof.

Examples of 6-membered, unsaturated or partially saturated, monocyclic hetero ring containing as hetero atoms, two or three nitrogen atoms, represented by CyB-(2) are pyridazine, pyrimidine, pyrazine, triazine, and partially saturated rings thereof.

Examples of 6-membered, unsaturated or partially saturated, monocyclic hetero ring containing as hetero atoms, one nitrogen atom, represented by CyB-(3), are pyridine, dihydropyridine, and tetrahydropyridine.

Examples of 4- or 5-membered, unsaturated or partially saturated, monocyclic hetero ring containing as hetero atoms, one, two or three nitrogen atoms, represented by CyB-(4), are pyrrole, imidazole, pyrazole, triazole, azetine, and partially saturated rings thereof,

Examples of 4-7 membered, unsaturated or partially saturated, monocyclic hetero ring containing as hetero atoms, one or two oxygen atoms, or one or two sulfur atoms represented by CyB-(5), are thiophene, furan, thiain, pyran, dithiain, dioxin, dioxole, and partially saturated rings thereof.

Preferably, such compounds are administered without therapeutic amounts of an NSAID.

The present invention is also a method of treating mammals with precancerous lesions by administering a pharmacologically effective amount of an pharmaceutical composition (preferably enterically coated) that includes compounds of Formula I. In still another form, the invention is a method of inducing apoptosis in human cells by exposing those cells to an effective amount of compounds of formula I, wherein R₁ through R₆ to those cells sensitive to such a compound.

As used herein, the term “precancerous lesion” includes syndromes represented by abnormal neoplastic, including dysplastic, changes of tissue. Examples include adenomatous growths in colonic, breast or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

As used herein, the term “carcinomas” refers to lesions that are cancerous. Examples include malignant melanomas, breast cancer, and colon cancer.

As used herein, the term “neoplasm” refers to both precancerous and cancerous lesions as well as hyperplastic conditions.

Compounds useful in the methods of this invention may be formulated into compositions together with pharmaceutically acceptable carriers for oral administration in solid or liquid form, or for rectal or topical administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for rectal administration are preferably suppositories that may contain, in addition to the compounds of Formula I, excipients such as cocoa butter or a suppository wax.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The pharmaceutical compositions of this invention are preferably packaged in a container (e.g. a box or bottle, or both) with suitable printed material (e.g. a package insert) containing indications, directions for use, etc.

The synthesis of compounds useful in practicing this invention is described in U.S. Pat. No. 5,439,985 as shown in the following. According to the present invention, of the compounds of the present invention, the compounds of the formula:

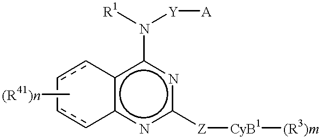

wherein R₄₁ is (1) hydrogen, (2) C1-4 alkyl, (3) C1-4 alkoxy, (4) —COOR₈, (5) —NR₉₁ R₁₀₁, in which R₉₁ is hydrogen, C1-4 alkyl or phenyl(C1-4 alkyl) and R₁₀₁, is hydrogen or C1-4 alkyl, provided that both R₉₁ and R₁₀₁ are not hydrogen, (6) SO₂NR₉ R₁₀, in which R₉ and R₁₀ are as hereinbefore defined, (7) halogen, (8) trifluoromethyl, (9) nitro, (10) cyano, (11) C1-4 alkylthio, (12) tri(C1-4 alkyl) silylethynyl, (13) —SO₂N═CHNR₁₁ R₁₀, in which R₁₁ and R₁₀ are the same meaning as hereinbefore defined, or (14) —CONR₁₂ R₁₃, in which R₁₂ and R₁₃ are the same meaning as hereinbefore defined, CyB₁ is as hereinbefore defined for CyB, provided that a carbon atom in the ring should bond to Z, and the other symbols are as hereinbefore defined; and the compounds of the formula:

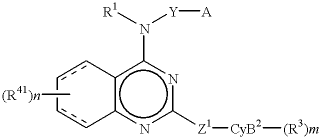

wherein Z_{1,} is single bond or methylene, CyB₂ is as hereinbefore defined for CyB, provided that a nitrogen atom in the ring should bond to Z₁, and the other symbols are as hereinbefore defined; may be prepared by using a series of reactions depicted in Scheme A and B, respectively, wherein R₅₀ is C1-4 alkyl and the other symbols are as hereinbefore defined.

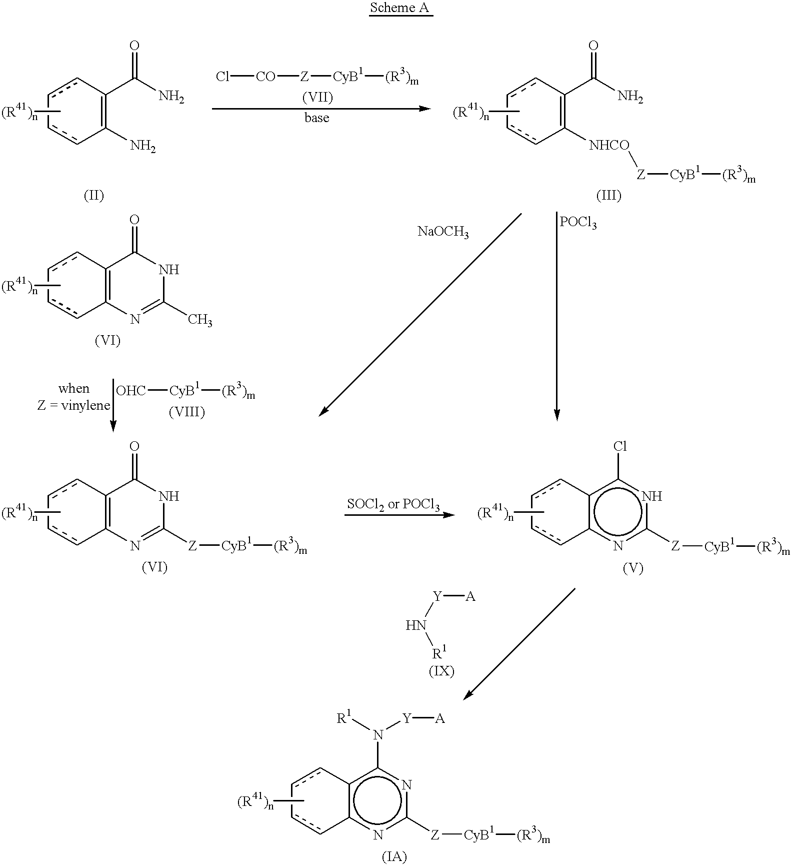

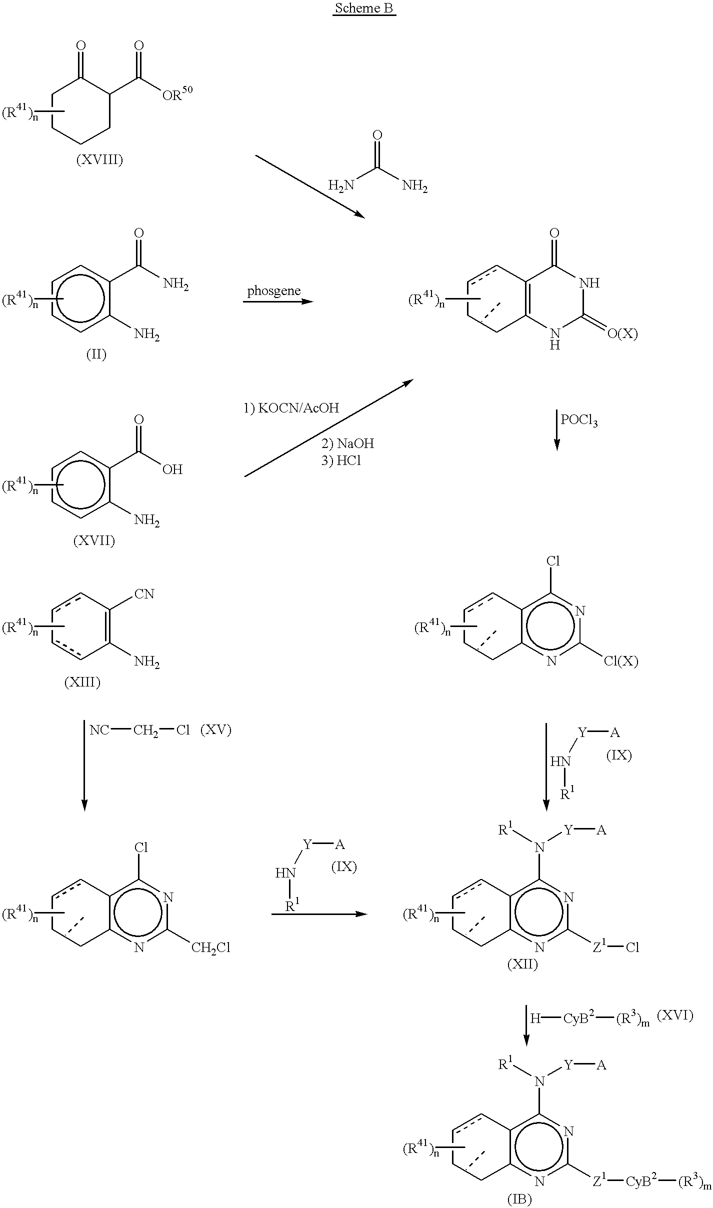

Each reaction in Scheme A and B may be carried out by methods known per se, under conditions described therein.

For example, the compounds of the formula (IA) may be prepared from those of Formula (V) by the reaction with an amine of Formula (IX) in a proper organic solvent such as a lower alkanol (e.g. ethanol) or tetrahydrofuran, or a mixture thereof, at a temperature from ambient to reflux, for several hours to several days, if necessary in the presence of a base such as triethylamine.

Further, the compounds of the formula (IB) may be prepared from those of the formula (XII) by the reaction with a cyclic amine of Formula (XVI) in phenol at a reflux temperature for several hours.

Furthermore, the compounds of the present invention, of Formula:

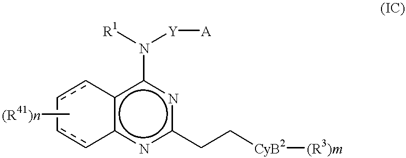

wherein the various symbols are as defined above; may be prepared from those of Formula:

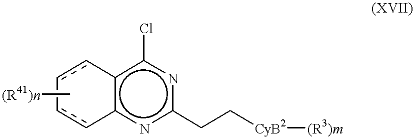

wherein the various symbols are as defined previously; by the methods described above for the conversion of the compounds of Formula (V) into those of Formula (IA). The compounds of Formula (XVII) may be prepared by the methods similar to those described in Scheme A.

On the other hand, the compounds of Formula I other than those of Formulae (IA), (IB) and (IC) may be prepared by the methods known per se described below.

The compounds of Formula (I) wherein R₄ is amino may be prepared from those wherein R₄ is nitro, by the reduction with zinc etc. in a proper organic solvent.

The compounds of Formula (I) wherein R₄ is hydroxy may be prepared from those wherein R₄ is alkoxy such as methoxy, by the reaction with hydrogen bromide or tribromoboron.

The compounds of Formula (I) wherein R₄ is —NHCOR₁₁, wherein R₁₁ is as defined above, may be prepared from those wherein R₄ is nitro, by the reaction with the corresponding organic acid such as acetic acid in the presence of zinc dust.

The compounds of Formula (I) wherein R₄ is NHSO₂R₁₁, wherein R₁₁ is as defined above, may be prepared from those wherein R4 is amino by the reaction with the corresponding alkylsulfonyl chloride such as methanesulfonyl chloride.

The compounds of Formula (I) wherein R₄ is —OCOR₁₁, wherein R₁₁ is as hereinbefore defined, may be prepared from those wherein R₄ is hydroxy by the esterification with the corresponding organic acid such as acetic acid.

The compounds of Formula (I) wherein R₄ is C1-4 alkylsulfinyl or C1-4 alkylsulfonyl may be prepared from those wherein R₄ is C1-4 alkylthio by the oxidation by oxidating agent such as hydrogen peroxide.

The compounds of Formula (I) wherein R₄ is hydroxymethyl may be prepared from those wherein R₄ is alkyoxycarbonyl, by the reduction with reducing agent such as lithium borohydride, lithium aluminum hydride etc.

The compounds of Formula (I) wherein R₄ is ethynyl may be prepared from those wherein R₄ is tri(C1-4 alkyl)silylethynyl, by the removal reaction of silyl group with tetrabutylammonium halide.

The compounds of Formula (I) wherein R₄ is acetyl may be prepared from those wherein R₄ is ethynyl, by the reaction with mercury sulfate and acetic acid in an acidic condition.

In each reaction in the present specification, products may be purified by conventional manner. For example, it may be carried out by distillation at atmospheric or reduced pressure, high performance liquid chromatography, thin layer chromatography or column chromatography using silica gel or magnesium silicate, washing or recrystallization. Purification may be carried out after each reaction, or after a series of reactions.

The starting materials of Formulae (II), (VI) and (XIII), and each reagents of Formulae (VII), (VIII), (IX), (XV), (XVI), (XVII) and (XVIII) used in the process for the preparation of the present invention are known per se or may be easily prepared by known methods.

Examples of representative compounds of the present invention are listed as follows:

1 4-(2-(2-hydroxyethoxy)ethyl)amino-2-(6-methyl-3 -pyrdyl)quinazoline,

2 4-(2-(2-hydroxyethoxy)ethyl)amino-2-(6-methoxy-3-pyridyl)quinazoline,

3 4-(2-(2-hydroxyethoxy)ethyl)amino-2-(6-chloro-3 -pyridyl)quinazoline,

4 4-(2-(2-hydroxyethoxy)ethyl)amino-2-(6-trifluoromethyl-3 -pyridyl)quinazoline,

5 4-(2-(2-hydroxyethoxy)ethyl)amino-6-methyl-2-(3-pyridyl)quinazoline,

6 4-(2-(2-hydroxyethoxy)ethyl)amino-6-methoxy-2-(3-pyridyl)quinazoline,

7 4-(2-(2-hydroxyethoxy)ethyl)amino-6,7-dimethoxy-2-(3-pyridyl)quinazoline,

8 4-(2-(2-hydroxyethoxy)ethyl)amino-6-carboxy-2-(3-pyridyl)quinazoline,

9 4-(2-(2-hydroxyethoxy)ethyl)amino-6-methoxycarbon yl-2-(3-pyridyl)quinazoline,

10 4-(2-(2-hydroxyethoxy)ethyl)amino-6-amino-2-(3-pyridyl)quinazoline,

11 4-(2-(2-hydroxyethoxy)ethyl)amino-6-(N,N-dimethylamino)-2-(3-pyridyl) quinazoline,

12 4-(2-(2-hydroxyethoxy)ethyl)amino-6-acetylamino-2-(3-pyridyl)quinazoline,

13 4-(2-(2-hydroxyethoxy)ethyl)amino-6-methanesulfonylamino-2-(3-pyridyl) quinazoline,

14 4-(2-(2-hydroxyethoxy)ethyl)amino-6-sulfamoyl-2-(3-pyridyl)quinazoline,

15 4-(2-(2-hydroxyethoxy)ethyl)amino-6-acetoxy-2-(3-pyridyl)quinazoline,

16 4-(2-(2-hydroxyethoxy)ethyl)amino-6-chloro-2-(3-pyridyl)quinazoline,

17 4-(2-(2-hydroxyethoxy)ethyl)amino-6-bromo-2-(3-pyridyl)quinazoline,

18 4-(2-(2-hydroxyethoxy)ethyl)amino-7-fluoro-2-(3-pyridyl)quinazoline,

19 4-(2-(2-hydroxyethoxy)ethyl)amino-6-trifiuoromethyl-2-(3-pyridyl)quinazoline,

20 4-(2-(2-hydroxyethoxy)ethyl)amino-6-hydroxy-2-(3-pyridyl)quinazoline,

21 4-(2-(2-hydroxyethoxy)ethyl)amino-6-nitro-2-(3-pyridyl)quinazoline,

22 4-(2-(2-hydroxyethoxy)ethyl)amino-6-cyano-2-(3-pyridyl)quinazoline,

23 4-(2-(2-hydroxyethoxy)ethyl)amino-6-ethynyl-2-(3-pyridyl)quinazoline,

24 4-(2-(2-hydroxyethoxy)ethyl)amino-6-methyl-2-(4-pyridyl)quinazoline,

25 4-(2-(2-hydroxyethoxy)ethyl)amino-6-methoxy-2-(4-pyridyl)quinazoline,

26 4-(2-(2-hydroxyethoxy)ethyl)amino-6,7-dimethoxy-2-(4-pyridyl)quinazoline,

27 4-(2-(2-hydroxyethoxy)ethyl)amino-6-carboxy-2-(4-pyridyl)quinazoline,

28 4-(2-(2-hydroxyethoxy)ethyl)amino-6-methoxycarbonyl-2-(4-pyridyl)quinazoline,

29 4-(2-(2-hydroxyethoxy)ethyl)amino-6-amino-2-(4-pyridyl)quinazoline,

30 4-(2-(2-hydroxyethoxy)ethyl)amino-6-(N,N-dimethylainino)-2-(4-pyridyl) quinazoline,

31 4-(2-(2-hydroxyethoxy)ethyl)amino-6-acetylamino-2-(4-pyridyl)quinazoline,

32 4-(2-(2-hydroxyethoxy)ethyl)amino-6-methanesulfonylamino-2-(4-pyridyl) quinazoline,

33 4-(2-(2-hydroxyethoxy)ethyl)amino-6-sulfamoyl-2-(4-pyridyl)quinazoline,

34 4-(2-(2-hydroxyethoxytethyl)amino-6-acetoxy-2-(4-pyridyl)quinazoline,

35 4-(2-(2-hydroxyethoxy)ethyl)amino-6-chloro-2-(4-pyridyl)quinazoline,

36 4-(2-(2-hydroxyethoxy)ethyl)amino-6-bromo-2-(4-pyridyl)quinazoline,

37 4-(2-(2-hydroxyethoxy)ethyl)amino-7-fluoro-2-(4-pyridyl)quinazoline,

38 4-(2-(2-hydroxyethoxy)ethyl)amino-6-trifluoromethyl-2-(4-pyridyl)quinazoline,

39 4-(2-(2-hydroxyethoxy)ethyl)amino-6-hydroxy-2-(4-pyridyl)quinazoline,

40 4-(2-(2-hydroxyethoxy)ethyl)amino-6-nitro-2-(4-pyridyl)quinazoline,

41 4-(2-(2-hydroxyethoxy)ethyl)amino-6-cyano-2-(4-pyridyl)quinazoline,

42 4-(2-(2-hydroxyethoxy)ethyl)amino-6-ethynyl-2-(4-pyridyl)quinazoline,

43 4-(2-(2-hydroxyethoxy)ethyl)amino-6-methyl-2-(1-imidazolyl)quinazoline,

44 4-(2-(2-hydroxyethoxy)ethyl)amino-6-methoxy-2-(1 -imidazolyl)quinazoline,

45 4-(2-(2-hydroxyethoxy)ethyl)amino-6,7-dimethoxy-2-(1-imidazolyl)quinazoline,

46 4-(2-(2-hydroxyethoxy)ethyl)amino-6-carboxy-2-(1-imidazolyl)quinazoline,

47 4-(2-(2-hydroxyethoxy)ethyl)amino-6-methoxycarbonyl-2-(1-imidazolyl)quinazoline,

48 4-(2-(2-hydroxyethoxy)ethyl)amino-6-amino-2-(1-imidazolyl)quinazoline,

49 4-(2-(2-hydroxyethoxy)ethyl)amino-6-(N,N-dimethylamino)-2-(1-imidazolyl) quinazoline,

50 4-(2-(2-hydroxyethoxy)ethyl)amino-6-acetylamino-2-(1-imidazolyl)quinazoline

51 4-(2-(2-hydroxyethoxy)ethyl)amino-6-methanesulfonylamino-2-(1-imidazolyl) quinazoline

52 4-(2-(2-hydroxyethoxy)ethyl)amino-6-sulfamoyl-2-(1-imidazolyl)quinazoline,

53 4-(2-(2-hydroxyethoxy)ethyl)amino-6-acetoxy-2-(1-imidazolyl)quinazoline,

54 4-(2-(2-hydroxyethoxy)ethyl)amino-6-bromo-2-(1-imidazolyl)quinazoline,

55 4-(2-(2-hydroxyethoxy)ethyl)amino-6-iodo-2-(1-imidazolyl)quinazoline,

56 4-(2-(2-hydroxyethoxy)ethyl)amino-7-fluoro-2-(1-imidazolyl)quinazoline,

57 4-(2-(2-hydroxyethoxy)ethyl)amino-6-trifluoromethyl-2-(1-imidazolyl)quinazoline,

58 4-(2-(2-hydroxyethoxy)ethyl)amino-6-hydroxy-2-(1-imidazolyl)quinazoline,

59 4-(2-(2-hydroxyethoxy)ethyl)amino-6-nitro-2-(1-imidazolyl)quinazoline,

60 4-(2-(2-hydroxyethoxy)ethyl)amino-6-cyano-2-(1-imidazolyl)quinazoline,

61 4-(2-(2-hydroxyethoxy)ethyl)amino-2-(2-azepinyl)quinazoline,

62 4-(2-(2-hydroxyethoxy)ethyl)amino-2-(1,5-diazepin-2-yl)quinazoline,

63 4-(2-(2-hydroxyethoxy)ethyl)amino-2-(2-pyrimidinyl)quinazoline,

64 4-(2-(2-hydroxyethoxy)ethyl)amino-2-(2-triazinyl)quinazoline,

65 4-(2-(2-hydroxyethoxy)ethyl)amino-2-(2-pyridyl)quinazoline,

66 4-(2-(2-hydroxyethoxy)ethyl)amino-2-(4-pyridyl)quinazoline,

67 4-(2-(2-hydroxyethoxy)ethyl)amino-2-(2-(3-pyridyl)ethyl)quinazoline,

68 4-(2-(2-hydroxyethoxy)ethyl)amino-2-(2-(3-pyridyl)vinyl)quinazoline,

69 4-(2-(2-hydroxyethoxy)ethyl)amino-2-(2-pyrrolyl)quinazoline,

70 4-(2-(2-hydroxyethoxy)ethyl)amino-2-(2-ethyl-1-imidazolyl)quinazoline,

71 4-(2-(2-hydroxyethoxy)ethyl)amino-2-((1-imidazolyl)methyl)quinazoline,

72 4-(2-(2-hydroxyethoxy)ethyl)amino-2-(2-methyl-1-imidazolyl)quinazoline,

73 4-(2-(2-hydroxyethoxy)ethyl)amino-2-(1-triazolyl)quinazoline,

74 4-(2-(2-hydroxyethoxy)ethyl)amino-2-(2-thienyl)quinazoline,

75 4-(2-(2-hydroxyethoxy)ethyl)amino-2-(2-furyl)quinazoline,

76 4-(2-(2-hydroxyethylthio)ethyl)amino-2-(2-azepinyl)quinazoline,

77 4-(2-(2-hydroxyethylthio)ethyl)amino-2-(1,5-diazepin-2-yl)quinazoline,

78 4-(2-(2-hydroxyethylthio)ethyl)amino-2-(2-pyrimidinyl)quinazoline,

79 4-(2-(2-hydroxyethylthio)ethyl)amino-2-(2-triazinyl)quinazoline,

80 4-(2-(2-hydroxyethylthio)ethyl)amino-2-(2-pyridyl)quinazoline,

81 4-(2-(2-hydroxyethylthio)ethyl)amino-2-(4-pyridyl)quinazoline,

82 4-(2-(2-hydroxyethylthio)ethyl)amino-2-(2-(3-pyridyl)ethyl)quinazoline,

83 4-(2-(2-hydroxyethylthio)ethyl)amino-2-(2-(3-pyridyl)vinyl)quinazoline,

84 4-(2-(2-hydroxyethylthio)ethyl)amino-2-(2-pyrrolyl)quinazoline,

85 4-(2-(2-hydroxyethylthio)ethyl)amino-2-(1-imidazolyl)quinazoline,

86 4-(2-(2-hydroxyethylthio)ethyl)amino-2-((1-imidazolyl)methyl)quinazoline

87 4-(2-(2-hydroxyethylthio)ethyl)amino-2-(2-methyl-1-imidazolyl)quinazoline,

88 4-(2-(2-hydroxyethylthio)ethyl)amino-2-(1-triazolyl)quinazoline,

89 4-(2-(2-hydroxyethylthio)ethyl)amino-2-(2-thienyl)quinazoline,

90 4-(2-(2-hydroxyethylthio)ethyl)amino-2-(2-furyl)quinazoline,

91 4-(2-(2-hydroxyethoxy)ethyl)amino-6-methoxy-2-((3-pyridyl)methyl)quinazoline,

92 4-(2-(2-hydroxyethoxy)ethyl)amino-6-chloro-2-((3-pyridyl)methyl)quinazoline,

93 4-(2-(2-hydroxyethoxy)ethyl)amino-6-ethynyl-2-((3-pyridyl)methyl)quinazoline,

94 4-(2-(2-hydroxyethoxy)ethyl)amino-6-methoxycarbonyl-2-((3-pyridyl)methyl) quinazoline,

95 4-(2-(2-hydroxyethoxy)ethyl)amino-6-methoxy-2-(2-(3-pyridyl)ethyl)quinazoline,

96 4-(2-(2-hydroxyethoxy)ethyl)amino-6-chloro-2-(2-(3-pyridyl)ethyl)quinazoline,

97 4-(2-(2-hydroxyethoxy)ethyl)amino-6-ethynyl-2-(2-(3-pyridyl)ethyl)quinazoline,

98 4-(2-(2-hydroxyethoxy)ethyl)amino-6-methoxycarbonyl-2-(2-(3-pyridyl)ethyl) quinazoline,

99 4-(2-(2-hydroxyethoxy)ethyl)amino-6-methoxy-2-(2-(3-pyridyl)vinyl)quinazoline,

100 4-(2-(2-hydroxyethoxy)ethyl)amino-6-chloro-2-(2-(3-pyridyl)vinyl)quinazoline,

101 4-(2-(2-hydroxyethoxy)ethyl)amino-6-ethynyl-2-(2-(3-pyridyl)vinyl)quinazoline,

102 4-(2-(2-hydroxyethoxy)ethyl)amino-6-methoxycarbonyl-2-(2-(3-pyridyl)vinyl) quinazoline,

103 4-(2-(2-hydroxyethoxy)ethyl)amino-6-methoxy-2-((1-imidazolyl)methyl) quinazoline,

104 4-(2-(2-hydroxyethoxy)ethyl)amino-6-chloro-2-((1-imidazolyl)methyl)quinazoline,

105 4-(2-(2-hydroxyethoxy)ethyl)amino-6-ethynyl-2-((1-imidazolyl)methyl)quinazoline,

106 4-(2-(2-hydroxyethoxy)ethyl)amino-6-methoxycarbonyl-2-((1-imidazolyl)methyl) quinazoline,

107 4-(2-(2-hydroxyethoxy)ethyl)amino-6-methoxy-2-(2-(1-imidazolyl)ethyl) quinazoline,

108 4-(2-(2-hydroxyethoxy)ethyl)amino-6-chloro-2-(2-(1-imidazolyl)ethyl)quinazoline,

109 4-(2-(2-hydroxyethoxy)ethyl)amino-6-ethynyl-2-(2-(1-imidazolyl)ethyl) quinazoline,

110 4-(2-(2-hydroxyethoxy)ethyl)amino-6-methoxycarbonyl-2-(2-(1-imidazolyl)ethyl) quinazoline,

111 4-(2-(2-hydroxyethylsulfinyl)ethyl)amino-2-(2-azepinyl)quinazoline,

112 4-(2-(2-hydroxyethylsulfinyl)ethyl)amino-2-(1,5-diazepin-2-yl)quinazoline,

113 4-(2-(2-hydroxyethylsulfinyl)ethyl)amino-2-(2-pyrimidinyl)quinazoline,

114 4-(2-(2-hydroxyethylsulfinyl)ethyl)amino-2-(2-triazinyl)quinazoline,

115 4-(2-(2-hydroxyethylsulfinyl)ethyl)amino-2-(2-pyridyl)quinazoline,

116 4-(2-(2-hydroxyethylsulfinyl)ethyl)amino-2-(4-pyridyl)quinazoline,

117 4-(2-(2-hydroxyethylsulfinyl)ethyl)amino-2-(2-(3-pyridyl)ethyl)quinazoline,

118 4-(2-(2-hydroxyethylsulfinyl)ethyl)amino-2-(2-(3-pyridyl)vinyl)quinazoline,

119 4-(2-(2-hydroxyethylsulfinyl)ethyl)amino-2-(2-pyrrolyl)quinazoline,

120 4-(2-(2-hydroxyethylsulfinyl)ethyl)amino-2-(1-imidazolyl)quinazoline,

121 4-(2-(2-hydroxyethylsulfinyl)ethyl)amino-2-((1-imidazolyl)methyl)quinazoline,

122 4-(2-(2-hydroxyethylsulfinyl)ethyl)amino-2-(2-methyl-1-imidazolyl)quinazoline,

123 4-(2-(2-hydroxyethylsulfinyl)ethyl)amino-2-(1-triazolyl)quinazoline,

124 4-(2-(2-hydroxyethylsulfinyl)ethyl)amino-2-(2-thienyl)quinazoline,

125 4-(2-(2-hydroxyethylsulfinyl)ethyl)amino-2-(2-furyl)quinazoline,

126 4-(2-(2-hydroxyethylsulfonyl)ethyl)amino-2-(2-azepinyl)quinazoline,

127 4-(2-(2-hydroxyethylsulfonyl)ethyl)amino-2-(1,5-diazepin-2-yl)quinazoline,

128 4-(2-(2-hydroxyethylsulfonyl)ethyl)amino-2-(2-pyrimidinyl)quinazoline,

129 4-(2-(2-hydroxyethylsulfonyl)ethyl)amino-2-(2-triazinyl)quinazoline,

130 4-(2-(2-hydroxyethylsulfonyl)ethyl)amino-2-(2-pyridyl)quinazoline,

131 4-(2-(2-hydroxyethylsulfonyl)ethyl)amino-2-(4-pyridyl)quinazoline,

132 4-(2-(2-hydroxyethylsulfonyl)ethyl)amino-2-(2-(3-pyridyl)ethyl)quinazoline,

133 4-(2-(2-hydroxyethylsulfonyl)ethyl)amino-2-(2-(3-pyridyl)vinyl)quinazoline,

134 4-(2-(2-hydroxyethylsulfonyl)ethyl)amino-2-(2-pyrrolyl)quinazoline,

135 4-(2-(2-hydroxyethylsulfonyl)ethyl)amino-2-(1-imidazolyl)quinazoline,

136 4-(2-(2-hydroxyethylsulfonyl)ethyl)amino-2-((1-imidazolyl)methyl)quinazoline,

137 4-(2-(2-hydroxyethylsulfonyl)ethyl)amino-2-(2-methyl-1-imidazolyl)quinazoline,

138 4-(2-(2-hydroxyethylsulfonyl)ethyl)amino-2-(1-triazolyl)quinazoline,

139 4-(2-(2-hydroxyethylsulfonyl)ethyl)amino-2-(2-thienyl)quinazoline,

140 4-(2-(2-hydroxyethylsulfonyl)ethyl)amino-2-(2-furyl)quinazoline,

and further those described in Examples below are also representative compounds of the present invention.

The compounds of Formula (I), if desired, may be converted into acid addition salts by known methods. Preferably, acid addition salts are non-toxic and water-soluble. The suitable acid addition salts are, for example, salts of an inorganic acid such as hydrochloric acid, hydrobromic acid, hydroiodic acid, sulfuric acid, phosphoric acid, nitric acid, or an organic acid such as acetic acid, lactic acid, tartaric acid, benzoic acid, citric acid, methanesulfonic acid, ethanesulfonic acid, benzenesulfonic acid, toluenesulfonic acid, isethionic acid, glucuronic acid and gluconic acid.

The compounds of Formula (I), if desired, may be converted into salts by known methods. Preferable, salts are non-toxic salts and water-soluble. The suitable salts are salts of alkaline metal (sodium, potassium etc.), salts of alkaline earth metal (calcium, magnesium etc.), ammonium salts, salts of pharmaceutically acceptable organic amine (tetramethylammonium, triethylamine, methylamine, dimethylamine, cyclopentylamine, phenylmethylamine, phenethylamine, piperidine, monoethanolamine, diethanolamine, tris(hydroxymethyl)methylamine, lysine, arginine, N-methyl-D-glucamine etc.).

Throughout the specification including claims, it may be easily understood by those skilled in the art, that the alkyl, alkoxy, groups include straight-chained and also branched-chained ones. Accordingly, all isomers produced by any differences in stereo configuration, such as asymmetric carbons are included in the present invention.

The doses to be administered are determined depending upon age, body weight, symptom, the desired therapeutic effect, the route of administration, and the duration of the treatment etc. In the human adult, the doses per person are generally between .06 mg and 3.7 mg by oral or parenteral administration, up to several times per day, or continuous administration between 1 and 24 hrs. per day intravenously.

As mentioned above, the doses to be used depend upon various conditions. Therefore, there are cases in which doses lower than or greater than the ranges specified above may be used.

The following examples from the above U.S. patent are intended to illustrate, but not limit, compounds useful in practicing the methods of the present invention.

### EXAMPLE 1

### 4-Fluoroisatoic Anhydride

To a solution of 2-amino-4-fluorobenzoic acid (4.65 g) in 50 ml of mixed solvent (10:1=toluene:tetrahydrofuran) is added phosgene (4.46 g, 1.93M solution of toluene ) dropwise via a drop funnel. The mixture is stirred at room temperature for 1 hour and then heated to reflux overnight. The mixture is concentrated to about 10 ml and cooled in refrigerator. The precipitate is filtered, washed with ether (5 ml×2) and air-dried to give the title compound (5.43 g) as a white solid having the following physical data.

NMR (200 MHz, DMSO-d6): delta 6.92 (dd, 1H), 7.11 (td, 1H), 8.00 (dd, 1H), 11.92 (broad, 1H).

### EXAMPLE 2

### 4-Fluoroanthranilamide

A solution of the isatoic anhydride compound (3.62 g, prepared in Example 1) in 100 ml of tetrahydrofuran is placed in a 200 ml round bottle equipped with gas in- and outlet. The anhydrous ammonia gas is gently bubbled into the solution for 1.5 to 2 hours. After removal of the solvent the residue is taken up in methylene chloride (30 ml) and water (30 ml). The precipitate is collected by filtration and washed with methylene chloride (10 ml) to give the title compound (1.95 g) as a pale white solid having the following physical data.

NMR (200 MHz, DMSO-d6): delta 6.70 (m, 1H), 6.82 (m, 1H), 6.90 (broad, 2H), 7.72 (m, 1H).

The following compounds are obtained by the same procedure as Example 1 and Example 2, by using the corresponding substituted anthranilic acid compound.

### EXAMPLE 2(a)

### 5-Methylanthranilamide

The product is collected by filtration as a pale solid.

NMR (200 MHz, DMSO-d6): delta 2.24 (s, 3H), 5.50 (broad, 2H), 6.62 (d, 1H), 7.07 (dd, 1H), 7.16 (d, 1H).

### EXAMPLE 2(b)

### 5-Chloroanthranilamide

The product is collected by filtration as a pale solid.

NMR (200 MHz, DMSO-d6): delta 5.68 (broad, 2H), 6.64 (d, 1H), 7.20 (dd, 1H), 7.35 (d, 1H).

### EXAMPLE 2(c)

### 5-Bromoanthranilamide

The product is collected by filtration as a pale brown.

NMR (200 MHz, DMSO-d6): delta 6.66 (dd,1 H), 6.72 (broad, 2H), 7.20 (broad, 1H), 7.26 (dt, 1H), 7.70 (t, 1H), 7.82 (broad, 1H).

### EXAMPLE 2(d)

### 5-Nitroanthranilamide

The product is collected by filtration as a solid.

NMR (200 MHz, DMSO-d6): delta 6.80 (dd,1 H), 7.40 (broad, 1H), 7.90 (broad, 2H), 8.03 (dt, 1H), 8.20 (broad, 1H), 8.56 (t, 1H).

### EXAMPLE 3

### 4-Fluoro-2-[N-(3-Pyridylcarbonyl)Amino]Benzamide

To a solution of the anthranilamide compound (1.54 g, prepared in Example 2) and triethylamine (1.4 g) in 100 ml of tetrahydrofuran is added nicotinoyl chloride hydrochloride (1.95 g). The resulting mixture is heated to reflux for one to three days and then concentrated. The residue is taken up in water (25 ml) and chloroform (30 ml). The insoluble crude product is collected by filtration and then vacuum dried. The crude product is triturated with 10 ml of ether and pentane solution (1:1) to afford the title compound (2.27 g) as a white solid having the following physical data.

NMR (200 MHz, DMSO-d6): delta 7.10 (td, 1H), 7.80 (m, 1H), 7.99 (broad, 1H), 8.07 (m, 1H), 8.40-8.55 (m, 3H), 8.90 (m, 1H), 9.15 (m, 1H).

### EXAMPLE 4

### 7-Fluoro-2-(3 -Pyridyl)Quinazoiln-4-One

To a suspension of the benzamide compound (1.6 g, prepared in Example 3) in 60 ml of toluene is added sodium methoxide (853 mg). The solution is heated to reflux for one to three days. After cooling to room temperature, the mixture is quenched with ammonium chloride solution (30 ml) with a vigorously shaking. The mixture is cooled in refrigerator and the insoluble product is collected by filtration and dried in vacuum to give the title compound (1.39 g) as a white solid having the following physical data.

NMR (200 MHz, DMSO-d6): delta 7.43 (td, 1H), 7.53-7.64 (m, 2H), 8.20-8.28 (m, 1H), 8.50 (dt, 1H), 8.78 (dd, 1H), 9.29 (m, 1H).

### EXAMPLE 5

### 4-Chloro-7-Fluoro-2-(3-Pyridyl)Quinazoline Hydrochloride

A suspension of the quinazolinone compound (1.2 g, prepared in Example 4) in 20 ml of thionyl chloride is heated to reflux for three hours. The excess of thionyl chloride is removed by distillation. The residue is distilled azeotropically with benzene (5 ml×3) and then reduced the total volume to about 5 ml. After cooling in refrigerator, precipitate is collected by filtration and washed with benzene twice to give the title compound (1.38 g) as a crystalline solid having the following physical data.

NMR (200 MHz, DMSO-d6): delta 7.80-7.95 (m, 2H), 8.07 (dd, 1H), 8.43-8.49 (m, 1H), 8.95 (d, 1H), 9.06 (dr, 1H), 9.65 (m, 1H).

The following compounds are obtained by the same procedure as Example 3 arrow right Example 4 arrow right Example 5, by using the anthranilamide compound prepared in Examples 2(a), 2(b) or 2(c), or being on sale, and the corresponding acid chloride.

### EXAMPLE 5(a)

### 4-Chloro-6-Methyl-2-(3-Pyridyl)Quinazoline Hydrochloride

The product is collected by filtration as a white solid.

NMR (200 MHz, DMSO-d6): delta 2.62 (s, 3H), 7.96-8.14 (m, 4H), 8.98 (d, 1H), 9.16 (d, 1H), 9.63 (m, 1H).

### EXAMPLE 5(b)

### 4,6-Dichloro-2-(3-Pyridyl)Quinazoline Hydrochloride

The product is collected by filtration as a white solid.

mp: 210°-214° C.

NMR (CDC13): delta 7.28-8.17 (m, 3H), 8.35 (m, 1H), 8.89 (dd, 1H), 9.55 (dt, 1H), 9.98 (d, 1H).

### EXAMPLE 5(c)

### 4-Chloro-6,7-Dimethoxy-2-(3-Pyridyl)Quinazoline Hydrochloride

The product is collected by filtration as a white solid.

NMR (200 MHz, DMSO-d6): delta 4.04 (s, 3H), 4.06 (s, 3H), 7.46 (s, 1H), 7.56 (s, 1H), 7.95 (m, 1H), 8.93 (d, 1H), 9.09 (d, 1H), 9.60 (m, 1H).

### EXAMPLE 5(d)

### 4-Chloro-2-(2-Pyridyl)Quinazoline

The product is collected by filtration as a light brown powder.

mp: 120°-121° C.

### EXAMPLE 5(e)

### 6-bromo-4-Chloro-2-(3-Pyridyl)quinazoline Hydrochloride

NMR (200 MHz, DMSO-d6): delta 8.02 (m, 1H), 8.14 (dd, 1H), 8.33 (dt, 1H), 8.50 (t, 1H), 9,01 (d, 1H), 9.14(d, 1H), 9.64 (t, 1H).

### EXAMPLE 6

### 2-[N-(3-pyridylcarbonyl)amino]benzamide

To a solution of anthranilamide (8.2 g, being on sale) and triethylamine (18.0 g)in 100 ml of tetrahydrofuran/methylene chloride (1:1), is added nicotinoyl chloride hydrochloride (10.8 g). The mixture is allowed to stir at room temperature, under nitrogen atmosphere, for six hours. The solution is then concentrated under reduced pressure. The concentrate is taken up in ethyl acetate and water and the mixture filtered. The solid material is triturated in ether and filtered to give the title compound (11.5 g) as a yellow powder having the following physical data.

mp: 220°-222° C.

### EXAMPLE 7

### 2-(3 -Pyridyl)Quinazolin-4-One

To a solution of the benzamide compound (11.5 g, prepared in Reference example 6) in 100 ml of toluene is added 95% sodium methoxide (5.7 g). The solution is heated at 60°-80° C. for three hours under nitrogen atmosphere. After cooling to room temperature, the solution is diluted with ammonium chloride solution. After stirring for one-half hour, the mixture is filtered. An NMR of the filtered material indicated the reaction is incomplete. The material is taken up in toluene and ethanol and 95% sodium methoxide (5.7 g) is added. The resulting solution is heated to reflux and stirred via a mechanical stirrer, under nitrogen atmosphere, overnight. The solvent is thereby evaporated, and the concentrate in the flask is collected and washed with ammonium chloride solution and methylene chloride. The solid material is collected by filtration and allowed to dry to give the title compound as a gray powder having the following physical data.

mp: 275°-276° C.

NMR (200 MHz, DMSO-d6): delta 7.50-7.61 (m, 2H), 7.75-7.90 (m, 2), 8.16 (d, 1H), 8.49 (m, 1H), 8.77 (d, 1H), 9.31 (s, 1H).

IR (KBr): nu 3185 (w), 3045 (m), 2915 (w), 1677 (s), 1603 (m), 1558 (w), 1474 (m), 769 (m)cm₋₁.

### EXAMPLE 8

### 4-Chloro-2-(3-Pyridyl)Quinazoline

A solution of the quinazolinone compound (6.7 g, prepared in Reference example 7) and 5.7 ml of N,N-dimethylaniline in 200 ml of benzene is heated to reflux, under nitrogen atmosphere, for one-half hour with the removal of 15 ml of distillate. After cooling to room temperature, phosphorus oxychloride (4.5 g) is added and the resulting solution heated to reflux for six hours. After cooling to room temperature, the solution is washed with ice water and dilute sodium hydroxide solution. The organic extract Is dried over sodium sulfate and concentrated under reduced pressure. The concentrate is triturated in ether and collected to give the title compound (3.0 g) (mp: 178°-179° C.).

The following compounds are obtained by the same procedure as Example 6 arrow right Reference example 7 arrow right Example 8, by using anthranilamide and the corresponding acid chloride.

### EXAMPLE 8(a)

### 4-Chloro-2-(4-Pyridyl)Quinazoline

The product is collected by filtration as a brown solid.

mp: 158°-160° C.

### EXAMPLE 8(b)

### 4-Chloro-2-(2-Chloro-5-Pyridyl)Quinazoline

NMR (CDC13): delta 7.47 (d, 1H), 7.73 (t, 1H), 7.95 (t, 1H), 8.05-8.32 (m, 2H), 8.81 (dd, 1H), 9.55 (ds, 1H).

### EXAMPLE 8(c)

### 4-Chloro-2-(2-Thienyl)Quinazoline

The product is collected by filtration as a tan powder.

mp: 121°-124° C.

### EXAMPLE 8(d)

### 4-Chloro-2-(2-Furyl)Quinazoline

The product is collected by the filtration as a tan powder.

mp: 116°-119° C.

### EXAMPLE 9

### 5-Nitro-2-[N-(3-Pyridylcarbonyl)Amino]Benzamide

The title compound is obtained by the same procedure as Reference example 3, by using 5-nitroanthranilamide (prepared in Example 2 (d)).

The product is collected by filtration as a white solid.

NMR (200 MHz, DMSO-d6): delta 7.70 (m, 1H), 8.20 (broad, 1H), 8.35 (dt, 1H), 8.49 (dd, 1H), 8.85-8.92 (m, 3H), 9.15 (t, 1H).

### EXAMPLE 10

### 4-Chloro-6-Nitro-2-(3-Pyridyl)Quinazoline

A suspension of the benzamide compound (0.925 g, prepared in Reference example 9) in phosphorous oxychloride (6 ml) is heated to reflux for 16 hours. After cooling to room temperature, the mixture is diluted by chloroform (30 ml) and then poured into 30 ml of ice-water mixture. The mixture is cooled in ice bath and carefully neutralized to pH 8 with a temperature control under 10o C. The aqueous layer is extracted with chloroform (50 ml×3). Combined organic layers are dried over with potassium carbonate and concentrated under reduced pressure to give the title compound (0.8 g) having the following physical data.

NMR (CDCl₃): delta 7.27-7.35 (m, 2H), 7.52 (dd, 1 Hi, 8.46-8.63 (m, 3Hi, 8.87 (d, 1H), 9.42 (s, 1H).

### EXAMPLE 11

### 4-Phenylmethylamino-7-Fluoro-2-(3-Pyridyl)Quinazoline

To a warm solution of the 4-chloroquinazoline compound (1.18 g, prepared in Reference example 5) in 50 ml ethanol is added phenylmethylamine (2.00 g). The mixture is heated to reflux for sixteen hours. The solution is then concentrated, and the residue taken up in chloroform and ammonium chloride solution. The aqueous layer is extracted with chloroform (30 ml×3) and dried over sodium sulfate. After concentration, the residue is triturated in pentane/ether solution to give the title compound (0.88 g) as a pale white solid having the following physical data.

mp: 199°-203° C.

NMR (CDCl₃): delta 5.00 (d, 2H), 6.01 (broad, 1H), 7.20 (td, 1H), 7.25-7.50 (m, 6H), 7.55 (dd, 1H), 7.70-7.77 (m, 1H), 8.70 (dd, 1H), 8.79 (dt, 1H), 9.74 (m, 1H).

IR (KBr): nu 697 (s), 775 (s), 1166 (m), 1259 (m), 1341 (s), 1375 (s), 1444 (s), 1535 (s), 1592 (s), 1626 (s), 3135 (m), 3250 (m) cm₋₁.

### EXAMPLE 12

### 4-Phenylmethylamino-7-Fluoro-2-(3-Pyridyl)Quinazoline Dihydrochloride

To a suspension of the free base (0.70 g, prepared in Example 11) in 10 ml methanol is added excess amount of HCl in methanol. The mixture is stirred at room temperature for one-half hour. The solvent is removed, and the residue is triturated in ether (30 ml). The title compound (0.84 g) as a white powder having the following physical data, is obtained after filtration.

mp: 250° C.

NMR (CDCl3): delta 4.50 (d, 2H), 7.25-7.40 (m, 3H), 7.49-7.53 (m, 2H), 7.64 (dt, 1H), 7.82 (dd, 1H), 7.99 (m, 1H), 8.67 (m, 1H), 8.97 (dd, 1H), 9.15 (dd, 1H), 9.60 (d, 1H), 10.18 (broad, 1H).

IR (KBr): nu 704 (m), 1266 (m), 1457 (s), 1574 (s), 1632 (s), 2920-2440 (broad, s), 3115 (broad, s) cm⁻¹.

### EXAMPLE 13

The following compounds are obtained by the same procedure as Example 11, or Example 11 and Example 12, by using the corresponding 4-chloroquinazoline compound prepared by Examples 5, 5(a) to 5(e) or Example 8, 8(a) to 8(d) and the proper amine.

4-Phenylmethylamino-6-Methyl-2-(3-Pyridyl)Quinazoline And Its Salt

4-Phenylmethylamino-6-Chloro-2-(3-Pyridyl)Quinazoline And Its Dihydrochloride Salt

4-Phenylmethylamino-6,7-Dimethoxy-2-(3-Pyridyl)Quinazoline And Its Dihydrochloride Salt

4-Phenylmethylamino-2-(2-Pyridyl)Quinazoline And Its Dihydrochloride Salt

4-Phenylmethylamino-2-(3-Pyridyl)Quinazoline And Its Dihydrochloride Salt

4-Phenylamino-2-(3 -Pyridyl)Quinazoline

4-(3-Methoxycarbonylphenyl)Amino-2-(3 -Pyridyl)Quinazoline

4-(4-Carboxyphenylmethyl)Amino-2-(3-Pyridyl)Quinazoline

4-(2-Thienylmethyl)Amino-2-(3-Pyridyl)Quinazoline And Its Dihydrochloride Salt

4-(3-Chlorophenylmethyl)Amino-2-(3-Pyridyl)Quinazoline And Its Dihydrochloride Salt

4-(3-Pyridylmethyl)Amino-2-(3-Pyridyl)Quinazoline And Its Salt

4-(3,4-Dimethoxyphenylmethyl)Amino-2-(3-Pyridyl)Quinazoline And Its Dihydrochloride Salt

4-Phenylethylamino-2-(3-Pyridyl)Quinazoline And Its Dihydrochloride Salt

4-(3-Trifiuoromethylphenylmethyl) Amino-2-(3-Pyridyl)Quinazoline Dihydrochloride

4-(4-N,N-Dimethylamino)PhenylmethylAmino-2-(3-Pyridyl)Quinazoline Trihydrochloride

4-(4-Sulfamoylphenylmethyl)Amino-2-(3-Pyridyl)Quinazoline Dihydrochloride

4-Phenytmethylamino-2-(4-Pyridyl)Quinazoline And Its Dihydrochloride Salt

4-Phenylamino-2-(4-Pyridyl)Quinazoline

4-Phenylmethylamino-2-(2-Chloro-5-Pyridyl)Quinazoline

4-Phenylmethylamino-2-(2-Thienyl)Quinazoline

4-Phenylamino-2-(2-Thienyl)Quinazoline

4-Phenylmethylamino-2-(2-Furyl)Quinazoline

4-Phenylamino-2-(2-Furyl)Quinazoline

6-Chloro-4-(2-(1-Methyl-2-Pyrrolyl)Ethyl)Amino-2-(3-Pyridyl)Quinazoline And Its Dihydrochloride Salt

4-Phenylmethylamino-6-Bromo-2-(3-Pyridyl) Quinazoline And Its Dihydrochloride Salt

4-Phenylmethylamino-6-Nitro-2-(3-Pyridyl) Quinazoline And Its Dihydrochloride Salt

4-(Cyclopropylmethyl)Amino-2-(3-Pyridyl) Quinazoline And Its Dihydrochloride Salt

4-(3-Methylphenylmethyl)Amino-2-(3-Pyridyl)

4-(2-(1-Methyl-2-Pyrrolyl)Ethyl)Amino-2-(3-Pyridyl)Quinazoline

4-(3-Nitrophenylmethyl)Amino-2-(3-Pyridyl) Quinazoline And Its Dihydrochloride Salt

4-(5-Methyl-3-Isoxazolyl)Amino-2-(3-Pyridyl) Quinazoline And Its Dihydrochloride Salt

The following compounds are obtained by the same procedure as described in Examples 2, 3, 4 and 5 and Examples 11 and 12 or in Example 6, 7 and 8 and Examples 11 and 12, by using isatoic anhydride.

6-Iodo-4-Phenylmethylamino-2-(3-Pyridyl)Quinazoline Dihydrochloride

6-Fluoro-4-Phenylmethylamino-2-(3-Pyridyl)Quinazoline Dihydrochloride

4-(3-Carboxyphenyl)Amino-2-(4-Pyridyl)Quinazoline

### EXAMPLE 14

### 6-Acetylamino-4-Phenylmethylamino-2-(3-Pyridyl)Quinazoline

To warmed suspension of the nitroquinazoline compound (141 mg, prepared in Example 13(z)) in acetic acid (4 ml) is added zinc dust (80 mg). The red mixture is heated to reflux for overnight. After cooling down to room temperature, the precipitate is removed by filtration. The filtrate is neutralized to pH 8 and extracted with chloroform. The insoluble solid is removed by filtration during the extraction. The chloroform is dried over potassium carbonate and then concentrated to 0.5 ml (total volume). The precipitate is collected by filtration to give the title compound (20 mg).

### EXAMPLE 15

### 6-Chloro-(1h,3h)-Quinazolin-2,4-Dione

To a solution of 5-chloroanthranilamide (3.4 g) in tetrahydrofuran (50 ml) is added phosgene (16 ml, 1.93M solution in toluene) via an addition funnel. The reaction mixture is stirred at room temperature for 4 hours and then heated to reflux for another two hours. The reaction mixture is concentrated to a total volume about 10 ml. After cooling, the title compound (3.72 g) having the following physical data is collected by filtration and dried in vacuum.

### EXAMPLE 16

### 4-Chloro-2-Chloromethvlquinazoline

To a solution of anthranilonitrile (11.8 g) and chloroacetonitrile (7.5 g) in 1,4-dioxane (200 ml), cooled in an ice bath, is bubbled HCl gas. The reaction mixture is stirred for two and one-half hours at which time the reaction is allowed to warm to room temperature and continued to bubble HCl gas for 16 hours. After the HCl gas bubbling is ceased, nitrogen gas is bubbled through to remove any unreacted HCl gas. The mixture is concentrated at 45o C. in vacuo. The mixture is partitioned between methylene chloride (300 ml) and water (400 ml). The organic layer is separated, dried over anhydrous magnesium sulfate, and concentrated. The concentrate is dissolved in 200 ml of warm hexane, filtered and allowed to cool to room temperature. The title compound (9.1 g) is collected by filtration.

### EXAMPLE 17

### 2,4-Dichloroquinazoline

A mixture of benzoyleneurea (20.0 g), phosphorus oxychloride (100 ml) and N,N-dimethylaniline (12 ml) is,refluxed for five hours. After stirring overnight at room temperature, the mixture is heated to reflux once more for an additional four hours. The cooled mixture is then poured into ice and the precipitate collected. The precipitate is purified on silica gel column with 5% methanol/chloroform as eluent. The isolated product is triturated in ether/hexane and collected to obtain the title compound (6.9 g).

The following compound is obtained by the same procedure as Example 17, by using 6-chloro-(1H,3H)-quinazolin-2,4-dione prepared by Example 15: 2,4,6-Trichloroquinazoline.

### EXAMPLE 18

### 4-Phenylmethylamino-2-Chloroquinazoline

The title compound is obtained by the same procedure as Example 11, by using the dichloroquinazoline prepared in Example 17 and phenylmethylamine (equivalent to dichloroquinazoline).

### EXAMPLE 19

### 4-Phenylmethylamino-2-(1-Imidazolyl)Quinazoline

A mixture of the 4-phenylmethylamino-2-chloroquinazoline (0.81 g, prepared in Example 18), imidazole (0.81 g) and phenol (3.0 g) is heated to reflux for four and one-half hours. The mixture is then taken up in chloroform, washed twice with sodium hydroxide solution, dried over anhydrous potassium carbonate and concentrated. The concentrate is triturated in ether and collected to obtain the title compound (0.7 g) as a yellow solid.

The following compounds are obtained by the same procedure as Example 19, by using 4-phenylmethylamino-2-quinazoline prepared in Example 18 or corresponding quinazoline, and the proper heterocyclic compounds:

4-phenylmethylamino-2-(2-methyl-1-imidazolyl )quinazoline,

4-phenylmethylamino-2-( 1,2,4-triazol-1-yl)quinazoline,

4-phenylmethylamino-6-chloro-2-(1-imidazolyl)quinazoline,

4-phenylmethylamino-2-((1-imidazolyl)methyl)quinazoline, and

6-ethoxycarbonyl-4-phenylmethylamino-2-(1-imidazolyl)quinazoline

### EXAMPLE 20

### 4-Phenylmethylamino-2-(1-Imidazolyl)Quinazoline Dihydrochloride

The title compound is obtained by the same procedure as Example 12, by using the free base prepared in Example 19 and HCl/methanol solution.

By the same procedure as described in Example 17 and 18 and Example 19 and 20, the following compounds can be made:

4-phenylmethylamino-6-chloro-2-(1-imidazolyl)quinazoline dihydrochloride,

4-phenylmethylamino-2-((1-imidazolyl)methyl)quinazoline dihydrochloride.

The following compounds are obtained by the same procedure as described in Examples 17, 18 and Examples 19 and 20, by using the corresponding (1H,3H)-quinazoline-2,4-dione or its derivative and corresponding amine:

6-bromo-4-phenylmethylamino-2-(1-imidazolyl)quinazoline dihydrochloride,

7-chloro-4-phenylmethylamino-2-(1-imidazolyl)quinazoline,

6-chloro-4-phenylmethylamino-2-(1-imidazolylmethyl)quinazoline,

6-nitro-4-phenylmethylamino-2-(1-imidazolyl)quinazoline hydrochloride,

6-methoxy-4-phenylmethylamino-2-(1-imidazolyl)quinazoline dihydrochloride,

6-chloro-4-phenylamino-2-(1-imidazolylmethyl)quinazoline dihydrochloride,

6-chloro-4-(3-carboxyphenyl)amino-2-(1-imidazolylmethyl)quinazoline dihydrochloride,

6-dimethylaminosulfonyl-4-phenylmethylamino-2-(1-imidazolyl)quinazoline hydrochloride,

4-(2-furylmethyl)amino-2-(1-imidazolyl)quinazoline dihydrochloride,

4-(2-thienylmethyl)amino-2-(1-imidazolyl)quinazoline,

4-(2-tetrahydrofuranylmethyl)amino-2-(1-imidazolyl)quinazoline,

4-(2-methoxyethyl)amino-2-(1-imidazolyl)quinazoline dihydrochloride,

4-phenylmethylamino-2-(1-imidazolyl)-5,6,7,8-tetrahydro-quinazoline dihydrochloride,

6-dimethylaminomethylideneaminosulfonyl-4-phenylmethylamino-2-(1imidazolyl)quinazoline dihydrochloride,

6-(phenylmethylaminosulfonyl)-4-phenylmethylamino-2-(1-imidazolyl)quinazoline,

4-(2-phenylethyl)amino-2-(1-imidazolyl)quinazoline dihydrochloride,

4-cyclohexyl methylamino-2-(1-imidazolyl)quinazoline dihydrochloride,

6-carboxy-4-phenylmethylamino-2-(1-imidazolyl)-5,6,7,8-tetrahydroquinazoline

6-phenylmethylaminocarbonyl-4-phenylmethylamino-2-(1-imidazolyl)quinazoline dihydrochloride,

4-(4-tetrahdyropyranylmethyl)amino-2-(1-imidazolyl)quinazoline dihydrochloride,

6-methoxy-4-(4-tetrahydropyranylmethyl)amino-2-(1-imidazolyl)quinazoline dihydrochloride,

6-chloro-4-(4-tetrahydropyranylmethyl)amino-2-(1-imidazolyl)quinazoline dihydrochloride,

6-iodo-4-phenylmethylamino-2-(1-imidazolyl)quinazoline dihydrochloride,

4-(4-trifuloromethoxyphenylmethyl)amino-2-(1-imidazolyl)quinazoline dihydrochloride,

4-(3 -trifluoromethoxyphenylmethyl)amino-2-(1-imidazolyl)quinazoline dihydrochloride,

6-methoxy-4-(2-(2-hydroxyethoxy)ethyl)amino-2-(1-imidazolyl)quinazoline dihydrochloride,

4-(2-methoxyethyl)amino-2-(1-imidazolyl)-5,6,7,8-tetrahydroquinazoline dihydrochloride,

4-(2-methoxyethyl)amino-6-iodo-2-(1-imidazolyl)quinazoline dihydrochloride,

4-phenylmethylamino-6,8-diiodo-2-(1-imidazolyl)quinazoline dihydrochloride,,

4-(2-methoxyethyl)amino-6-methoxy-2-(2-methyl-1-imidazolyl)quinazoline dihydrochloride,

4-(2-hydroxyethyl)amino-6-methoxy-2-(1-imidazolyl)quinazoline dihydrochloride,

4-(2-methoxyethyl)amino-6,8-diiodo-2-(1-i midazolyl)quinazoline dihydrochloride,

4-(2-(2-hydroxyethoxy)ethyl)amino-2-(1 -imidazolyl)-5,6,7,8-tetrahydroquinazoline dihydrochloride,

4-(2-phenoxyethyl)amino-6-methoxy-2-(1-imidazolyl)quinazoline and its dihydrochloride,

4-(2-(2-hydroxyethoxy)ethyl)amino-6-iodo-2-(1-imidazolyl)quinazoline and its dihydrochloride,

4-(2-methoxyethyl)amino-6-methylthio-2-(1-imidazolyl)quinazoline and its dihydrochloride salt,

4-(2-(2-hydroxyethoxy)ethyl)amino-6-methylthio-2-(1-imidazolyl)quinazoline,

4-(2-(2-hydroxyethoxy)ethyl)amino-6-methylthio-2-(1-imidazolyl)quinazoline dihydrochloride,

6-.methylthio-4-phenylmethylamino-2-(1imidazolyl)quinazoline dihydrochloride,

4-(3-methoxypropyl)amino-6-methoxy-2-(1imidazolyl) quinazoline dihydrochloride,

4-(2-methoxyethyl)amino-6 -methoxycarbonyl-2-(1-imidazolyl)quinazoline,

4-[2-(2-hydroxyethoxy )ethyl]amino-6-methoxycarbonyl-2-(1-imidazolyl) quinazoline,

4-(2-methylthioethyl)amino-6-methoxy o-2-1-imidazolyl)quinazoline,

4-(2-methylsulfinylethyl)amino-6-methoxy-2-(1-imidazolyl)quinazoline,

4-(2-methylsulfonylethyl)amino-6-methoxy-2-(1-imidazolyl)quinazoline.

### EXAMPLE 21

### 2-(2-(3-Pyridyl)Vinyl)Quinazolin-4-One

A mixture of 2-methylquinazolin-4-one (6.1 g) and 3-pyridinecarbaldehyde (4.1 g) in acetic acid (80 ml) is heated to reflux for 20 hours. After cooling to room temperature, the precipitate is collected by filtration, ished with methanol and dried to obtain the title compound as an acetic acid salt (10.5 g).

### EXAMPLE 22

### 4-Chloro-2-(2-(3-Pyridyl)Vinyl)Quinazoline

A suspension of the quinazolinone compound (2.9 g, prepared in Example 21) in thionyl chloride (25 ml) and a few drops of dimethylformamide is heated at reflux for three hours. The mixture is then concentrated, the concentrate poured into 150 ml portions of chloroform, dried over potassium carbonate and concentrated to obtain the title compound (1.1 g) as a red oil.

### EXAMPLE 23

### 4-Phenylmethylamino-2-(2-(3-Pyridyl)Vinyl)Quinazoline

The title compound is obtained by the same procedure as Example 11, by using the 4-chloro compound prepared in Example 22 and phenylmethylamine. The product is purified by column chromatography.

mp: 178°-179° C.

NMR(CDCl3): delta 4.96 (d, 2H), 6.11 (broad, 1H), 7.30-7.55 .(m, 8H), 7.70-7.81 (m, 2H), 7.99 (d, 1H), 8.34 (s, 1H), 8.36-e.45 (m, 1H), 8.55-8.5e (dd, 1H), 8.9-8.91 (d, 1H).

IR (KBr): nu 3300 (m), 1577 (s), 1528 (s), 1434 (m), 1378 (s), 763 (m), 699 (m) cm₁.

### EXAMPLE 24

### 6-Ethoxycarbonyl-4-Phenylmethylamino-2-(1 -Imidazolyl)-5 6,7, 8-Tetrahydroquinazoline

To 349 mg (1.0 mmol) of 4-phemylmethylamino-2-(1-imidazolyl) quinazoline dihydrochloride prepared in Example 20 dissolved in 20 ml of tetrahydrofuran is added 0.4 ml of thionyl chloride. Initially, a white precipitate formed, but gradually all dissolved. After stirring for 15 minutes, 20 ml of ethanol is added. After stirring an additional 15 minutes, the mixture is concentrated, the concentrate triturated in ether and collected. The solid is found to be very hygroscopic, is taken up in chloroform, treated with potassium carbonate solution, separated, dried over anhydrous magnesium sulfate and concentrated. 278 mg (0.7 mmol, 73% yield) of the desired product is obtained as a white solid (free base).

mp: 196°-198° C.

NMR (DMSO- d6 ): delta 1.30 (t, 3H), 1.90 (m, 1H), 2.28 (m, 1H), 2.60 (m, 2H), 2.82 (m, 3H), 4,23 (q, 2H), 4.77 (d, 2H), 5.12 (m, 1H), 7.10 (s, 1H), 7.37 (m, 5H), 7.83 (s, 1H), 8.54 (s, 1H).

IR (KBr): 3245 (w), 1725 (ms), 1605 (s), 1532 (w), 1473 (m), 1426 (m), 1333 (w) cm₋₁.

To obtain the dihydrochloride salt of the title compound, a suspension of 240 mg (0.64 mmol) of the compound prepared above in 5 ml of ethanol is added 2 ml of approximately equal to 10% HCl in methanol. All the material gradually dissolved. After ten minutes, the mixture is concentrated in vacuo, triturated in ether and filtered to obtain 229 mg (0.51 mmol) of the desired product. (2HCl salt)

mp: 158°-161° C.

N (200 MHz, DMSO-d6) delta: 1.22 (t, 3H), 1.87(m, 1H), 2.14 (m, 1H), 2.55-3.00 (m, 5H), 7.79 (s, 1H), 8.23 (s, 1H), 9.77 (s, 1H).

IR (KBr) nu: 3225, 1718, 1642, 1612, 1518, 1393 cm₋₁.

### EXAMPLE 25

### 6-Ethylaminocarbonyl-4-Phenylmethylamino-2-(1 -Imidazolyl)-5,6,7,8-Tetrahydroquinazoline Dihydrochloride

By the same procedure as described in Example 24, by using ethylamine instead of ethanol, the title compound having the following physical data is given.

mp: 147° C., (dec.)

NMR (200 MHz, DMSO-d6) delta: 1.04 (q, 3H), 1.65-2.06 (m, 2H), 2.50-2.80 (m, 5H), 3.10 (m, 2H), 4.72 (m, 2H), 7.18-7.48 (m, 5H), 7.81 (s, 1H), 8.05 (t, 1H), 8.18 (M, 1H), 8.24 (m, 1H), 9.82 (s, 1H).

IR (K-Br) nu: 3265-2580, 2365, 1653, 1613, 1576, 1540, 1449, 1390, 1352, 1144, 1060, 750, 701,624 cm⁻¹.

### EXAMPLE 26

### 4-Phenylmethylamino-2-(1-Imidazolyl)Quinazoline Dimethanesulfonate

By the same procedure as described in Examples 17 and 18 and Example 19 and 20, by using methanesulfonic acid instead of hydrochloric acid, the title compound and the following compounds are given.

6,7-dimethoxy-4-phenylmethylamino-2-(1-imidazolyl)quinazoline dimethanesulfonate,

4-(3,4-dimethoxyphenyl methyl)amino-2-(1-imidazolyl)quinazoline 1.5 methanesulfonate, and 4-(2-phenoxyethyl)amino-2-(1-imidazolyl)quinazoline dimethanesulfonate.

### EXAMPLE 27

### 6-Carboxy-4-Phenylmethylamino-2-(1-Imidazolyl)-5,6,7,8-Tetrahydroquinazoline Sodium Salt

A solution of 200 mg (0.57 mmol) of 6-carboxy-4-phenylmethylamino-2-(1-imidazolyl)-5,6,7,8-tetrahydroquinolazine dihydrochloride prepared in Example 20 dissolved in 25 ml of tetrahydrofuran is filtered to remove dark insoluble material present. To the filtrate is added 0.25 ml (0.62 mmol) of 2.5N sodium hydroxide solution. Some precipitate formed. The mixture is concentrated and pumped in vacuum. The concentrate is triturated in tetrahydrofuran and ether and filtered. The solid is washed with ether and filtered to obtain 190 mg (0.51 mmol) of the desired product as a white solid. mp: 240o C., (dec.) NMR (200 MHz, DMSO-d6) delta: 1.50-1.82 (m, 2H), 1.88-2.35 (m, 2H), 2.59 (m, 3H), 4.62 (s, 2H), 6.98 (s, 1H), 7.12-7.48 (m, 5H), 7.73 (s, 1H), 7.86 (m, 1H), 8.33 (s, 1H).

By the same procedure as described in Example 27, 6-carboxy-4-phenylmethylamino-2-(1-imidazolyl)quinazoline sodium salt can also be obtained.

### EXAMPLE 28

### 4-(11-Dimethyl-2-Methoxyethyl)Amino-2-Chloroquinazoline

A mixture of 2,4-dichloroquinazoline (995 mg, 5 mmol), triethylamine (0.7 ml, 5 mmol) and 1,1-dimethyl-2-methoxyethylamine (30 ml, 0.5M methanol sol., 15 mmol) is stood at room temperature for 1 week. The reaction mixture is concentrated and partitioned between ethyl acetate and water. The organic layer is washed with water and brine, dried over MgSO₄ and concentrated. The residue is purified on 50 g of silica gel column eluting with 50% ethyl acetate in hexane to obtain the title compound (176 mg) as a white solid.

NMR (CDCl₃): delta 1.60 (s, 6H), 3.46 (s, 3H), 3.56 (s, 2H), 7.38-7.80 (m, 4H).

### EXAMPLE 29

### 4-(1,1-Dimethyl-2-Methoxyethyl) Amino-2-(1-Imidazolyl)Quinazoline Dihydrochloride

A mixture of the compound prepared in Example 28 (165 mg, 0.62 mmol), imidazole (169 mg, 2.48 mmol) and phenol (0.7 g) is heated at 150° C. for 40 min. After cooling, the reaction mixture is diluted with ethyl acetate, and washed with lN KOH and brine, and dried over MgSO₄. The filtrate is concentrated to leave a viscous oil, which is purified on 8 g of silica gel column eluting with 50% ethyl acetate in hexane to obtain the title compound (165 mg, 90% yield) as a colorless amorphous. (free base)

NMR (CDCl₃): delta 1.65 (s, 6H), 3.48 (s, 3H), 3.58 (s, 2H), 6.32 (broad, 1H), 7.17 (s, 1H), 7.40 (m, 1H), 7.62-7.81 (m, 3H), 7.97 (s, 1H), 8.67 (s, 1H).

To a solution of the compound above (160 mg, 0.54 mmol) in methanol (2 ml) is added excess HCI-methanol solution (2 ml). After stirring for 20 min at room temperature, the reaction mixture is concentrated. Excess HCI is evaporated with methanol (×3) to leave a white solid. Trituration with ether gives the HCl salt (185 mg) as a white powder. (HCl salt) (mp: 223°-225° C.).

NMR (200 MHz, DMSO-d6) delta: 9.80 (s, 1H), 8.59 (m, 1H), 8.34 (m, 1H), 7.84-7.96 (m, 3H), 7.78 (m, 1H), 7.60 (m, 1H), 3.78 (s, 2H), 3.29 (s, 3H), 1.57 (s, 6H). IR(KBr) nu: 1633, 1610, 1562,1520, 1474, 1397, 1108, 754 cm⁻¹.

By the same procedure as described in Example 28 and Example 29, by using corresponding amine, the following compounds can be made:

6-methoxy-4-(2-methoxyethyl)amino-2-(1-imidazolyl)quinazoline dihydrochloride; 6-chloro-4-(2-methoxyethyl)amino-2-(1-imidazolyl)quinazoline dihydrochloride; 4-(3-ethoxypropyl)amino-2-(1-imidazolyl)quinazoline dihydrochloride; 6-nitro-4-(2-methoxyethyl)amino-2-(1-imidazolyl)quinazoline hydrochloride; 6-chloro-4-(2-(2-hydroxyethoxy)ethyl)amino-2-(1-imidazolyl)quinazoline dihydrochloride; and 6,7-dimethoxy-4-(2-methoxyethyl)amino-2-(1-imidazolyl)quinazoline dihydrochloride.

### EXAMPLE 30

### 6-Chloro-4-(2-Ethoxyethyl)Amino-2-(3-Pyridyl)Quinazoline

A solution of 2-(3-pyridyl)-4,6-dichloroquinazoline (1.0 g, 3.2 mmol, prepared in Example 5(b)) and 2-methoxyethylamine (0.53 g, 7.0 mmol) in 50 ml of ethanol is heated to reflux overnight. The solution is concentrated, taken up in chloroform and water. After some mixing, the water layer is found to be slightly acidic and is basified with sodium carbonate. The mixture is then agitated and separated. The organic layer is dried over potassium carbonate and concentrated. The concentrate is purified on silica gel column with 5% methanol in chloroform as eluent. The product obtained is combined with additional material filtered from the aqueous layer, for a total of 0.35 g (1.1 mmol) of the title compound.

mp: 210°-212° C.

NMR (200 MHz, DMSO- d6 ): delta 3.32 (s, 3H), 3.67 (t, 211), 3.87 (qd, 2H), 7.53 (m, 1H), 7.82 (s, 2H), 8.48 (s, 1H), 8.71 (m, 3H), 9.59 (s, 1H)

IR (KBr): nu 3250 (m), 1692 (s), 1535 (s), 1430 (w), 1412 (w), 1366 (m), 1140 (m), 823 (m) cm⁻¹.

To a mixture of 0.35 g (1.1 mmol) of the compound prepared above in 5 ml of methanol is added 0.5 ml of 10% HCI in methanol. The solution is concentrated to 1 ml, triturated in ether, filtered and dried under vacuum. Obtained 0.33 g (0.85 mmol) of the hydrochloride salt (mp: 190o C., (dec.)).

NMR (200 MHz, DMSO-d6) delta: 3.32 (s, 3H), 3.71 (t, 2H), 3.94 (m, 2H), 8.01 (m, 2H), 8.12 (d, 1H), 8.75 (m, 1H), 9.01 (d, 1H), 9.20 (d, 1H), 9.66 (s, 1H).

IR (KBr) nu: 3425, 2500-3050, 1633, 1610, 1569, 1387, 1107 cm⁻¹.

By the same procedure as described in Example 30, by using corresponding amine, the following compound can be obtained:

6-chloro-4-(2-dimethylaminoethyl)amino-2-(3-pyridyl)quinazoline trihydrochloride.

### EXAMPLE 31

### 6-Hydroxy-4-Phenylmethylamino-2-(1-Imidazolyl)Quinazoline.

To 66 mg (0.2 mmol) of 6-methoxy-4-phenylmethylamino-2-(1-imidazolyl)quinazoline dihyrochloride prepared in Example 20 in 1 ml of acetic acid is added 0.8 ml (7 mmol) of 48% HBr in water. The mixture is heated below reflux for 23 hours then heated to full reflux for four hours. After cooling to room temperature, 15 ml of water is added to the solution, and the precipitate is filtered and dried under vacuum. The material is purified on a preparative silica gel plate with 10% methanol in chloroform. Obtained 13 mg (41 mu mol) of the desired product as a solid. mp: 230° C., (dec.)

NMR (200 MHz, CD3OD) delta: 4.86 (s, 2H), 7.05 (s, 1H), 7.15-7.38 (m, 4H), 7.40-7.50 (m, 3H), 7.58-7.66 (m, 1H), 7.92 (s, 1H), 8.52 (s, 1H).

IR (KBr) nu: 3370, 3030, 2365, 1749, 1710, 1653, 1596, 1559, 1523, 1488, 1465, 1407, 1376, 1291, 1244, 1162, 1098, 1060, 911,831 cm⁻¹.

By the same procedure as described in Example 30, the hydrochloride of the title compound having the following physical data is given. (2HCl salt)

mp: 1550 C., (dec.)

NMR (200 MHz, DMSO-d6) delta: 4.92 (m, 2H), 7.22-7.77 (m, 8H), 7.86 (s, 1H), 8.38 (s, 1H), 9.36 (m, 1H), 9.94 (s, 1H).

IR (KBr) nu: 3395-2640, 2365, 1734, 1628, 1607, 1567, 1542, 1473, 1361, 1353, 1289, 1260, 1201, 1107, 1015, 835, 753, 702 cm⁻¹.

### EXAMPLE 32

### 4-(2-(2-Hydroxyethoxy)Ethyl)Amino-6-Methylsulfinyl-2-(1-Imidazolyl) Quinazoline And Its Dihydrochloride

To 1.38 g of the 4-(2-(2-hydroxyethoxy)ethyl)amino-6-methylsulfinyl-2-(1-imidazolyl) quinazoline prepared in Example 20 dissolved in 10 ml of acetic acid is added 4 ml of 30% hydrogen peroxide. The reaction is monitored by TLC. After stirring for 1/2 hour, the mixture is poured into 15 g of 50% w/w sodium hydroxide and ice. The resulting mixture is extracted four times with chloroform, dried over anhydrous magnesium sulfate and concentrated. The concentrate is triturated in ether and collected to obtain 1.26 g of the desired product as a white solid.

To 400 mg of the compound prepared above in 10 ml of methanol is added 1 ml of 10% HCl in methanol. After ten minutes, the mixture is concentrated, triturated in ether and the solid collected to yield 441 mg of the desired product as a dihyrochloride salt. (mp: 144°-147° C.).

NMR (200 MHz, DMSO-d6): d 2.85 (s, 3H), 3.50 (m, 4H), 3.70-3.90 (m, 4H), 4.59 (m, 1H), 7.11 (s, 1H), 7.82 (m, 1H), 7.98 (s, 1H), 8.02 (m, 1H), 8.62 (s, 1H), 8.67 (m, 1H), 9.14 (t, 1H). (2HCl salt) (mp: 190°-192° C.).

NMR (200 MHz, DMSO-d6): d 2.89 (s, 3H), 3.51 (s, 4H), 3.76 (m, 2H), 3.89(m, 2H), 7.90 (m, 2H), 8.14 (m, 1H), 8.45 (m, 1H), 8.89 (m, 1H), 9.62 (t, 1H), 10.10 (m, 1H).

By the same procedure as described in Example 32, by using corresponding thioether, the following compounds can be obtained:

4-(2-methoxyethyl)amino-6-methylsulfinyl-2-(1-imidazolyl)quinazoline and its dihydrochloride, and 6-methylsulfinyl-4-phenylmethylamino-2-(1-imidazolyl)quinazoline dihydrochloride.

### EXAMPLE 33

### 4-(2-Methoxyethyl)Amino-6-Methylsulfonyl-2-(1-Imidazolyl)Quinazoline Hydrochloride

To 0.63 g of the compound prepared in 4-(2-methoxyethyl)amino-6-methylthio-2-(1-imidazolyl)quinazoline prepared in accordance with Example 20 (free base) in 7 ml of acetic acid is added 3 ml of 30% hydrogen peroxide solution and the mixture is stirred at room temperature for 17 hours. The mixture is then poured into a solution of 50% w/w sodium hydroxide in ice. The resulting mixture is extracted twice with 70 ml portions of chloroform, dried over anhydrous magnesium sulfate and concentrated. The concentrate is triturated in ether, and the solid collected to obtain 0.36 g of the desired product as a white powder.

To a suspension of 300 mg of the compound above in 15 ml of methanol is added 1 ml of 10% HCl in methanol. The mixture becomes clear, then a precipitate formed. The mixture is concentrated to approximately 5 ml, diluted with ether and filtered to obtain 319 mg of the desired product as a white solid.

mp: 241o-243o C. (HCI salt)

mp: 226o-228o C.

NMR (200 MHz, DMSO-d6): d 3.32(s, 3H), 3.36(s, 3H), 3.67(m, 2H), 3.93(m, 2H), 7.81(s, 1H), 7.93(m, 1H), 8.30(m, 1I), 8.42(s, 1H), 9.16(m, 1H), 9.72(t, 1H), 9.92(s, 1 H).

By the same procedure as described in Example 33, 6-methylsulfonyl-4-phenylmethylamino-2-(1-imidazolyl)quinazoline hydrochloride can be obtained.

mp: 125o-130o C.

NMR (200 MHz, DMSO-d6): delta 3.34(s, 3H), 4.97(d, 2H), 7.31-7.50(m, 5H), 7.85(s, 1H), 7.93(d, 1H), 8.32(d, 1H), 8.44(s, 1H), 9.14(s, 1H), 9.98(s, 1H), 10.12(t, 1H).

IR (KBr): nu 3230(s), 3040(s), 2705(s), 2370(m), 1616(s), 1572(s), 1524(s), 1497(m), 1399(s), 1326(s), 1258(m), 1204(w), 1147(s), 1008(m), 834(w), 783(s), 730(w), 620(w), 535(m)cm⁻¹.

### EXAMPLE 34

### 6-Hydroxymethyl-4-Phenylmethylamino-2-(1-Imidazolyl)Quinazoline

To a suspension of 0.68 g of 6-ethoxycarbonyl-4-phenylmethylamino-2-(1-imidazolyl)quinazoline the compound prepared in Example 19 in 50 ml of anhydrous tetrahydrofuran is added 2 ml of 2M lithium borohydride in tetrahydrofuran. The reaction mixture is heated at reflux for two days. The mixture is then concentrated, diluted with water and the basic solution is acidified with 1N hydrochloric acid. The resulting solution is then basified with potassium carbonate, filtered and the solid washed with water and allowed to dry. The solid material is purified on silica gel column eluting with 5% methanol in chloroform, yielding 85 mg of the desired product.

mp: 173o C. (dec.).

NMR (200 MHz, DMSO-d6): delta 4.67(d, 1H), 4.90(d, 1H), 5.47(t, 1H), 7.23(m, 1H), 7.25-7.51(m, 5H), 7.67-7.85(m, 2H), 8.12(m, lH), 8.34(m, 1H), 8.9 (s, H), 9.5 H).

IR (KBr): nu 3445(mw), 2365(mw), 1599(s), 1559(m), 1505(mw), 1444(w), 1410(m), 1340(w), 1161(w), 1073(w)cm⁻¹.

By the same procedure as described in Example 34, the following compounds can be obtained.

4-(2-methoxyethyl)amino-6-hydroxymethyl-2-(1-imidazolyl)quinazoline, and

4-[2-(2-hydroxyethoxy)ethyl]amino-6-hydroxymethyl-2-(1-imidazolyl)quinazoline.

### EXAMPLE 35

### 6-Iodoquinazolin-2,4-Dione

To a mixture of 25.36 g of 2-amino-5-iodobenzoic acid in 250 ml of water and 90 ml of THF is added 7.40 g of glacial acetic acid and stirred at room temperature. Then 7.82 g of potassium cyanate in water dropwise, and the mixture is left overnight. Another 5.47 g of potassium cyanate is added, and the mixture is stirred overnight. A total of 160 g of NaOH pellets are added portionwise, keeping the mixture cool in ice-water bath. The mixture is stirred at room temperature overnight. The mixture is cooled in a refrigerator and the precipitate filtered through a sintered glass funnel. The precipitate is then dissolved in water and acidified with 4N HCl. The precipitate is collected by filtration. The solid is dried in a vacuum oven to yield 25.44 g of the title compound.

### EXAMPLE 36

### 6-(2-Triethylsilylethylnyl)Quinazolin-2,4-Dione

In a flask is placed 0.544 g of triphenylphosphine, 0.184 g of palladium chloride, and 5 ml of diethylamine. Stirred under a nitrogen atmosphere. To the resulting yellow mixture is added 75 ml of diethylamine, followed by 10.02 g of the compound prepared in Example 35. Then 19.8 mg of cuprous iodine is added to the purple suspension, which turns gray after 10 minutes. After 0.5 hr, 5.36 g of triethylsilyl acetylene and stirred at room temperature. After 3 hrs the solution turns purple. After another 1.5 hours, the solution turns brown and is left to stir overnight. The solvent is removed under reduced pressure at 40° C. and water is added. The mixture is acidified with 1N—HCI. The precipitated solid is collected by filtration, washed with water, and dried in a vacuum oven. The solid is then passed through a silica gel column, eluting with THF. After drying yielded 10.22 g of the title compound having the following physical data.

NMR (200 MHz, DMSO-d6): delta 0.65(dd, 6H), 0.93(dd, 9H), 7.15(d, 1H), 7.69(d, 1H), 11.38(br, 2H).

### EXAMPLE 37

### 2,4-Dichloro-6-(2-Triethylsilylethylnyl)(Quinazoline

To 5.09 g of the compound prepared in Example 36, is added 25 ml of POCl₃ and warmed. Then added 1.03 g of N,N-dimethylaniline and heated to reflux. After 3.5 hrs, the excess POCl₃ is removed under reduced pressure, and the residue diluted in chloroform and poured slowly over ice. The organic layer is collected and the solvent removed. The residue is passed through a silica gel column using 20% EtOAc/hexane as a solvent, yielding 1.4 g of the product having the following physical data.

NMR (200 MHz, CDCl3): delta 0.72(m, 6H), 1.00(m, 9H), 7.98(d, 1H), 8.33(s, 1H).

### EXAMPLE 38

### 2-Chloro-4-(2-Methoxyethyl)Amino-6-(2-Triethylsilylehnyl)Quinazoline

To 1.4 g of the compound prepared in Example 37 in 20 ml of chloroform is added 2-methoxyethylamine and stirred at room temperature for 1.5 hr. Then 4.2 ml of 1N—NaOH is added, the mixture is heated to reflux, and is left to reflux overnight. The solvent is removed under reduced pressure, and the residue taken up in chloroform and water. The organic layer is collected and dried over anhydrous potassium carbonate. Removal of solvent under reduced pressure yields 1.44 g of the title compound.

NMR (200 MHz, CDCl3): delta 0.73 (m, 6H), 1.07(m, 9H), 3.45(s, 3H), 3.69(t, 2H), 3.8a(dd, 2H), 6.32(br, 1H), 7.69(d, 1H), 7.7a(dd, 1H), 7.80(s, 1H).

### EXAMPLE 39

### 2-( -Imidazolyl)-4-(2-Methoxyethyl) Amino-6-(2-Triethylsilylethynyl) Quinazoline

To 1.32 g of the compound prepared in Example 38 in 5 ml of ethanol is added excess imidazole (0.93 g) and heated in an oil bath to 115° C. After 1.5 hours, the mixture is removed from heat and diluted in chloroform and washed with 1N—NaOH, collected the organic layer and washed with water. The organic layer is extracted and dried over anhydrous potassium carbonate. Removal or solvent yields 1.33 g of the title compound.

mp: 158°-160° C.

NMR (200 MHz, DMSO-d6): delta 0.70(q, 6H), 1.0S(t, 9H), 3.30(s, 3H), 3.64(t, 2H), 3.81 (dd, 2H), 7.10(s, 1H), 7.65(d, 1H), 7.78(dd, 1H), 7.96(s, 1H), 8.01 (s, 1H), 8.60(s, 1H), 8.95(br, 1H).

By the same procedures as described in Examples 35-38, and Example 39, the following compound is obtained: 2-(1-imidazolyl)-4-[2-(2-hydroxyethoxy)ethyl]amino-6-(2- triisopropylsilylethynyl)quinazoline

mp: 155°-156° C.;

NMR (200 MHz, CDCl3): delta 1.09 (s, 3H), 1.16 (s, 18H), 2.28 (br, 1H), 3.70 (m, 2H), 3.84 (dd, 4H), 3.95 (t, 2H), 6.65 (br, 1H), 7.14 (s, 1H), 7.68 (d, 1H), 7.75 (dd, 1H), 7.87 (s, 1H), 7.93 (s, 1H), 8.65 (s, 1H).

### EXAMPLE 40

### 6-Ethynyl-4-(2-Methoxyethyl)Amino-2-(1-Imidazolyl)Quinazoline

To 1.35 g of the compound prepared in Example 39 in 20 ml of THE is added 3.3 ml of tetrabutylammonium fluoride (1M in TIE), and is stirred at room temperature for 1.5 hrs. The excess THF is removed under reduced pressure, and the residue taken up in chloroform and water. The insoluble precipitate is collected by filtration, yielding 0.83 g of the title compound.

NMR (200 MHz, DMSO-d6): delta 3.33(s, 3H), 3.66(m, 2H), 3.83(m, 2H), 4.34(s, 1H), 7.11 (s, 1H), 7.65(d, 1H), 7.82(dd, 1H), 7.96(s, 1H), 8.57(d, 1H), 8.62(s, 1H), 8.90(broad, 1H).

IR (KBr): nu 3290(s), 2945(m), 1606(s), 1559(s), 1451(s), 1352(s), 1106(s), 835(s) cm₋₁.

By the same procedure, the following compound is given:

2-(1-imidazolyl)-4-[2-(2-hydroxyethoxy)ethyl]amino-6-ethynylquinazoline and its salt.

### EXAMPLE 41

### 6-Acetyl-4-(2-Methoxyethyl)Amino-2-(1-Imidazolyl)Quinazoline

To 0.541 g of the compound prepared in example 18 in 10 ml of acetic acid is added 0.7 ml of 10% H₂SO₄ and 0.10 g of mercury II sulfate and heated to reflux. After 2 hours, the solution is removed from heat and basified. The yellow precipitate is filtered. The solid is washed with THE and the solvent is removed under reduced pressure and the residue titrated in 50% ether/pentane. The solid is collected by filtration. Yielded 0.063 g of the desired product.

mp: 208°-210° C.

NMR (200 MHz, CDCl3): delta 2.64(s, 3H), 3.49(s, 3H), 3.79(t, 2H), 3.95(q, 2H), 7.00(broad, 1H), 7.16(t, 1H), 7.74(d, 1H), 7.95(t, 1H), 8.17(dd, 1H), 8.42(d, 1H), 8.67(t, 1H).

By the same procedure as described in Example 41, 4-[2-(2-hydroxyethoxy) ethyl]amino-6-acetyl-2-(1-imidazolyl)quinazoline can be obtained.

It will be understood that various changes and modifications can be made in the details of procedure, formulation and use without departing from the spirit of the invention, especially as defined in the,following claims.

We claim:

1. A method for inhibiting the growth of neoplastic cells comprising exposing the cells to a growth inhibiting effective amount of a compound of Formula I: 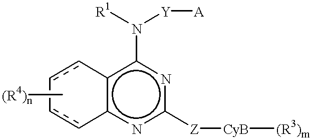
R₁ is hydrogen or C1-4 alkyl;
Y is C1-6 alkylene; A is —O—R₀ or —S(O)ₚ—R₀,
R₀ is C1-4 alkyl-hydroxy;
p is 0-2;
Z is single bond, methylene, ethylene (CH₂CH₂), vinylene (CH═CH) or ethynylene (C═C);
CyB is:
(1) 7-membered, unsaturated or partially saturated, monocyclic hetero ring containing as hetero atoms, one, two or three nitrogen atoms,
(2) 6-membered, unsaturated or partially saturated, monocyclic hetero ring containing as hetero atoms, two or three nitrogen atoms,
(3) 6-membered, unsaturated or partially saturated, monocyclic hetero ring containing as hetero atoms, one nitrogen atom,
(4) 4- or 5-membered, unsaturated or partially saturated, monocyclic hetero ring containing as hetero atoms, one, two or three nitrogen atoms, or
(5) 4-7 membered, unsaturated or partially saturated, monocyclic hetero ring containing as hetero atoms, one or two oxygen atoms, or one or two sulfur atoms;
R₃ is hydrogen, C1-4 alkyl, C1-4 alkoxy, halogen or trifluoromethyl;
R₄ is (1) hydrogen, (2) C1-4 alkyl, (3) C1-4 alkoxy, (4) —COOR₈, in which R₈ is hydrogen or C1-4 alkyl, (5) —NR₉R₁₀, (6) —NHCOR₁₁, (7) —NHSO₂R₁₁, (8) SO₂NR₉R₁₀, (9) —OCOR₁₁, (10) halogen, (11) trifluoromethyl, (12) hydroxy, (13) nitro, (14) cyano, (15) —SO₂N═CHN R₁₁R₁₀, (16) —CONR₁₂R₁₃, (17) C1-4 alkylthio, (18) C1-4 alkylsulfinyl, (19) C1-4 alkylsulfonyl, (20) ethynyl, (21) hydroxymethyl, (22) tri(C1-4 alkyl) silylethynyl or (23) acetyl; and m and n independently are 1 or 2; with the proviso that a CyB ring should not bond to Z through a nitrogen atom in the CyB ring when Z is vinylene or ethynylene;
R₉ is hydrogen, C1-4 alkyl or phenyl(C1-4 alkyl);
R₁₀ is hydrogen or C1-4 alkyl;
R₁₁ is C1-4 alkyl;
R₁₂ is hydrogen or C1-4 alkyl;
R₁₃ is C1-4 alkyl or phenyl(C1-4 alkyl); or pharmaceutically acceptable acid addition salts, pharmaceutically acceptable salts, or hydrates thereof.

2. The method of claim 1 wherein the compound is selected from the group consisting of 4-[2-(2-hydroxyethoxy)ethyl]amino-6-acetyl-2-(1-imidazolyl)quinazoline,
2-(1-imidazolyl)-4-[2-(2-hydroxyethoxy)ethyl]amino-6-ethynylquinazoline,
2-(1-imidazolyl)-4-[2-(2-hydroxyethoxy)ethyl]amino-6-(2-triisopropylsilylethynyl)quinazoline,
4-[2-(2-hydroxyethoxy)ethyl]amino-6-hydroxymethyl-2-(1-imidazolyl) quinazoline,
4-(2-(2-hydroxyethoxy)ethyl)amino-6-methylsulfinyl-2-(1-imidazolyl) quinazoline,
6-chloro-4-(2-(2-hydroxyethoxy)ethyl)amino-2-(1-imidazolyl)quinazoline,
4- [2-(2-hydroxyethoxy)ethyl] amino-6-methoxycarbonyl-2-(1-imidazolyl)quinazoline,
4-(2-(2-hydroxyethoxy)ethyl)amino-6-methylthio-2-(1-imidazolyl)quinazoline,
4-(2-(2-hydroxyethoxy)ethyl)amino-6-iodo-2-(1-imidazolyl)quinazoline,
4-(2-(2-hydroxyethoxy)ethyl)amino-2-(1-imidazolyl)-5,6,7,8-tetrahydroquinazoline or
6-methoxy-4-(2-(2-hydroxyethoxy)ethyl)amino-2-(1-imidazolyl)quinazoline,
and pharmaceutically acceptable acid addition salts thereof, pharmaceutically acceptable salts thereof, or hydrates thereof.

3. A method of treating a mammal having precancerous lesions comprising administering a pharmacologically effective amount of a compound of Formula I: 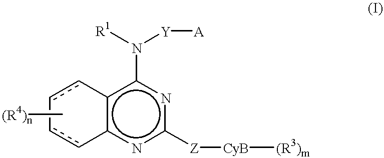
R₁ is hydrogen or C1 -4 alkyl;
Y is C1-6 alkylene; A is —O—R₀ or —S(O)ₚ—R₀,
R₀ is C1-4 alkyl-hydroxy;
p is 0-2;
Z is single bond, methylene, ethylene (CH₂CH₂), vinylene (CH═CH) or ethynylene (C═C);
CyB is:
(1) 7-membered, unsaturated or partially saturated, monocyclic hetero ring containing as hetero atoms, one, two or three nitrogen atoms,
(2) 6-membered, unsaturated or partially saturated, monocyclic hetero ring containing as hetero atoms, two or three nitrogen atoms,
(3) 6-membered, unsaturated or partially saturated, monocyclic hetero ring containing as hetero atoms, one nitrogen atom,
(4) 4- or 5-membered, unsaturated or partially saturated, monocyclic hetero ring containing as hetero atoms, one, two or three nitrogen atoms, or
(5) 4-7 membered, unsaturated or partially saturated, monocyclic hetero ring containing as hetero atoms, one or two oxygen atoms, or one or two sulfur atoms;
R₃ is hydrogen, C1-4 alkyl, C1-4 alkoxy, halogen or trifluoromethyl;
R₄ is (1) hydrogen, (2) C1-4 alkyl, (3) C1-4 alkoxy, (4) —COOR₈, in which R₈ is hydrogen or C1-4 alkyl, (5) —NR₉ R₁₀, (6) —NHCOR₁₁, (7) —NHSO₂R₁₁, (8) SO₂NR₉R₁₀, (9) —OCOR₁₁, (10) halogen, (11) trifluoromethyl, (12) hydroxy, (13) nitro, (14) cyano, (15) —SO₂N═CHN R₁₁R₁₀, (16) —CONR₁₂R₁₃, (17) C1-4 alkylthio, (18) C1-4 alkylsulfinyl, (19) C1-4 alkylsulfonyl, (20) ethynyl, (21) hydroxymethyl, (22) tri(C1-4 alkyl) silylethynyl or (23) acetyl; and m and n independently are 1 or 2; with the proviso that a CyB ring should not bond to Z through a nitrogen atom in the CyB ring when Z is vinylene or ethynylene;
R₉ is hydrogen, C1-4 alkyl or phenyl(C 1-4 alkyl);
R₁₀ is hydrogen or C1-4 alkyl;
R₁₁ is C1-4 alkyl;
R₁₂ is hydrogen or C1 -4 alkyl;
R₁₃ is C1-4 alkyl or phenyl(C1-4 alkyl);
or pharmaceutically acceptable acid addition salts, pharmaceutically acceptable salts, or hydrates thereof.

4. The method of claim 3 wherein the compound is selected from the group consisting of 4-[2-(2-hydroxyethoxy)ethyl]amino-6-acetyl-2-(1-imidazolyl)quinazoline,
2-(1-imidazolyl)-4-[2-(2-hydroxyethoxy)ethyl]amino-6-ethynylquinazoline,
2-(1-imidazolyl)-4-[2-(2-hydroxyethoxy)ethyl]amino-6-(2-triisopropylsilylethynyl)quinazoline,
4-[2-(2-hydroxyethoxy)ethyl]amino-6-hydroxymethyl-2-(1-imidazolyl) quinazoline,
4-(2-(2-hydroxyethoxy)ethyl)amino-6-methylsulfinyl-2-(1-imidazolyl) quinazoline,
6-chloro-4-(2-(2-hydroxyethoxy)ethyl)amino-2-(1-imidazolyl)quinazoline,
4-[2-(2-hydroxyethoxy)ethyl]amino-6-methoxycarbonyl-2-(1-imidazolyl)quinazoline,
4-(2-(2-hydroxyethoxy)ethyl)amino-6-methylthio-2-(1-imidazolyl)quinazoline,
4-(2-(2-hydroxyethoxy)ethyl)amino-6-iodo-2-(1-imidazolyl)quinazoline,
4-(2-(2-hydroxyethoxy)ethyl)amino-2-(1-imidazolyl)-5,6,7,8-tetrahydroquinazoline or
6-methoxy-4-(2-(2-hydroxyethoxy)ethyl)amino-2-(1-imidazolyl)quinazoline, and pharmaceutically acceptable acid addition salts thereof, pharmaceutically acceptable salts thereof, or hydrates thereof.

5. A method for regulating apoptosis in human cells comprising exposing said cells to an effective amount of a compound of Formula I: 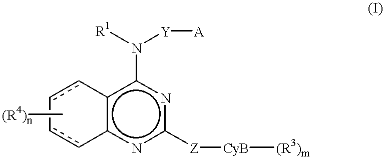
R₁ is hydrogen or C1-4 alkyl;
Y is C1-6 alkylene; A is —O—R₀ or —S(O)ₚ—R₀,
R₀ is C1-4 alkyl-hydroxy;
p is 0-2;
Z is single-bond, methylene, ethylene (CH₂CH₂), vinylene (CH═CH) or ethynylene (C═C);
CyB is:
(1) 7-membered, unsaturated or partially saturated, monocyclic hetero ring containing as hetero atoms, one, two or three nitrogen atoms,
(2) 6-membered, unsaturated or partially saturated, monocyclic hetero ring containing as hetero atoms, two or three nitrogen atoms,
(3) 6-membered, unsaturated or partially saturated, monocyclic hetero ring containing as hetero atoms, one nitrogen atom,
(4) 4- or 5-membered, unsaturated or partially saturated, monocyclic hetero ring containing as hetero atoms, one, two or three nitrogen atoms, or
(5) 4-7 membered, unsaturated or partially saturated, monocyclic hetero ring containing as hetero atoms, one or two oxygen atoms, or one or two sulfur atoms;
R₃ is hydrogen, C1 -4 alkyl, C1 -4 alkoxy, halogen or trifluoromethyl;
R₄ is (1) hydrogen, (2) C1-4 alkyl, (3) C1-4 alkoxy, (4) —COOR₈, in which R₈ is hydrogen or C1-4 alkyl, (5) —NR₉ R₁₀, (6) —NHCOR₁₁, (7) —NHSO₂R₁₁, (8) SO₂NR₉R₁₀, (9) —OCOR₁₁, (10) halogen, (11) trifluoromethyl, (12) hydroxy, (13) nitro, (14) cyano, (15) —SO₂N═CHN R₁₁R₁₀, (16) —CONR₁₂R₁₃, (17) C1-4 alkylthio, (18) C1-4 alkylsulfinyl, (19) C1-4 alkylsulfonyl, (20) ethynyl, (21) hydroxymethyl, (22) tri(C1-4 alkyl) silylethynyl or (23) acetyl; and m and n independently are 1 or 2; with the proviso that a CyB ring should not bond to Z through a nitrogen atom in the CyB ring when Z is vinylene or ethynylene;
R₉ is hydrogen, C1-4 alkyl or phenyl(C1-4 alkyl);
R₁₀ is hydrogen or C1-4 alkyl;
R₁₁ is C1-4 alkyl;
R₁₂ is hydrogen or C1-4 alkyl;
R₁₃ is C1-4 alkyl or phenyl(C1-4 alkyl); or pharmaceutically acceptable acid addition salts, pharmaceutically acceptable salts, or hydrates thereof.

6. The method of claim 5 wherein the compound is selected from the group consisting of: 4 -[2-(2-hydroxyethoxy)ethyl]amino-6-acetyl-2-(1-imidazolyl)quinazoline,
2-(1-imidazolyl)-4-[2-(2-hydroxyethoxy)ethyl]amino-6-ethynylquinazoline,
2-(1-imidazolyl)-4-[2-(2-hydroxyethoxy)ethyl]amino-6-(2-triisopropylsilylethynyl)quinazoline,
4-[2-(2-hydroxyethoxy)ethyl]amino-6-hydroxymethyl-2-(1-imidazolyl) quinazoline,
4-(2-(2-hydroxyethoxy)ethyl)amino-6-methylsulfinyl-2-(1-imidazolyl) quinazoline,
6-chloro-4-(2-(2-hydroxyethoxy)ethyl)amino-2-(1-imidazolyl)quinazoline,
4-[2-(2-hydroxyethoxy)ethyl]amino-6-methoxycarbonyl-2-(1-imidazolyl)quinazoline,
4-(2-(2-hydroxyethoxy)ethyl)amino-6-methylthio-2-(1-imidazolyl)quinazoline,
4-(2-(2-hydroxyethoxy)ethyl)amino-6-iodo-2-(1-imidazolyl)quinazoline,
4-(2-(2-hydroxyethoxy)ethyl)amino-2-(1-imidazolyl)-5,6,7, 8-tetrahydroquinazoline or
6-methoxy-4-(2-(2-hydroxyethoxy)ethyl)amino-²-(1-imidazolyl)quinazoline, and pharmaceutically acceptable acid addition salts thereof, pharmaceutically acceptable salts thereof, or hydrates thereof.
